(19) United States
(12) Patent Application Publication
(10) Pub. No.: US 2020/0202979 A1
(43) Pub. Date: Jun. 25, 2020

(54) NASAL-RELATED CHARACTERIZATION ASSOCIATED WITH THE NOSE MICROBIOME

(71) Applicant: PSOMAGEN, INC., Rockville, MD (US)

(72) Inventors: Zachary Apte, San Francisco, CA (US); Jessica Richman, San Francisco, CA (US); Daniel Almonacid, San Francisco, CA (US); Juan Ugalde, San Francisco, CA (US); Patricia Vera-Wolf, San Francisco, CA (US); Sandro Valenzuela, San Francisco, CA (US)

(21) Appl. No.: 16/642,618

(22) PCT Filed: Sep. 6, 2018
(86) PCT No.: PCT/US2018/049806

§ 371 Date: Feb. 27, 2020

Related U.S. Application Data
(60) Provisional application No. 62/554,985, filed on Sep. 6, 2017.

(51) Int. Cl. G16B 30/00; G16B 40/20; G16B 40/30; C12Q 1/689

(52) U.S. Cl. CPC G16B 30/00; C12Q 1/689; G16B 40/30; G16B 40/20

(57) ABSTRACT

Embodiments of a method and/or system (e.g., for nasal-related characterization) can include determining a microorganism dataset associated with a set of subjects; and/or performing a characterization process based on the microorganism dataset, where performing the characterization process can additionally or alternatively include performing a nasal-related characterization process, and/or determining one or more therapies, such as for one or more nasal-related conditions.

### CROSS-REFERENCE TO RELATED APPLICATIONS

This, application claims the benefit of U.S. Provisional Application Ser. No. 62/554,985 filed 6 Sep. 2017, which is incorporated in its entirety herein by this reference.

This application is related to U.S. application Ser. No. 15/606,743, filed 26 May 2017, which is a continuation of U.S. application Ser. No. 14/919,614, filed 21 Oct. 2015, which claims the benefit of U.S. Provisional Application Ser. No. 62/066,369 filed 21 Oct. 2014, U.S. Provisional Application Ser. No. 62/087,551 filed 4 Dec. 2014, U.S. Provisional Application Ser. No. 62/092,999 filed 17 Dec. 2014, U.S. Provisional Application Ser. No. 62/147,376 filed 14 Apr. 2015, U.S. Provisional Application Ser. No. 62/147,212 filed 14 Apr. 2015, U.S. Provisional Application Ser. No. 62/147,362 filed 14 Apr. 2015, U.S. Provisional Application Ser. No. 62/146,855 filed 13 Apr. 2015, and U.S Provisional Application Ser. No. 62/206,654 filed 18 Aug. 2015, which are each incorporated in its entirety herein by this reference.

### TECHNICAL FIELD

The disclosure generally relates to genomics and microbiology.

### BACKGROUND

A microbiome can include an ecological community of commensal, symbiotic, and pathogenic microorganisms that are associated with an organism. Characterization of the human microbiome is a complex process. The human microbiome includes over 10 times more microbial cells than human cells, but characterization of the human microbiome is still in nascent stages such as due to limitations in sample processing techniques, genetic analysis techniques, and resources for processing large amounts of data. Present knowledge has clearly established the role of microbiome associations with multiple health conditions, and has become an increasingly appreciated mediator of host genetic and environmental factors on human disease development. The microbiome is suspected to play at least a partial role in a number of health/disease-related states. Further, the microbiome may mediate effects of environmental factors on human, plant, and/or animal health. Given the profound implications of the microbiome in affecting a user's health, efforts related to the characterization of the microbiome, the generation of insights from the characterization, and/or the generation of therapeutics configured to rectify states of dysbiosis should be pursued.

Nasal passages are one of the main reservoirs of microbial diversity in the human body. An important characteristic of this reservoir is its permanent contact with the environment, where changes in the microbial composition can reflect geographical and temporal variations. Nasal passages also play a vital role in human health, being part of one of the first barriers of access to the human body.

Conventional approaches for analyzing the microbiomes (e.g., nose microbiomes) of humans, determining characterizations, and/or providing therapeutic measures based on gained insights have, however, left many questions unanswered.

### BRIEF DESCRIPTION OF THE FIGURES

FIG. 1A-1B include flowchart representations of variations of an embodiment of a method;

FIG. 2 includes variations of embodiments of a method and system;

FIG. 3A-3D include specific examples of taxa distribution in relation to calendar season and climate type;

FIG. 4 includes a flowchart representation of variations of an embodiment of a method;

FIG. 5 includes a variation of a process for generation of a characterization model in an embodiment of a method;

FIG. 6 includes variations of mechanisms by which probiotic-based therapies operate in an embodiment of a method;

FIG. 7 includes variations of sample processing in an embodiment of a method;

FIG. 8 includes examples of notification provision;

FIG. 9 includes a schematic representation of variations of an embodiment of the method;

FIG. 10A-10B includes variations of performing characterization processes with models;

FIG. 11 includes promoting a therapy in an embodiment of a method;

FIG. 12 includes a specific example of geographic locations, climate types, and number of samples associated with a set of samples.

### DESCRIPTION OF THE EMBODIMENTS

The following description of the embodiments is not intended to limit the embodiments, but rather to enable any person skilled in the art to make and use.

### 1. OVERVIEW

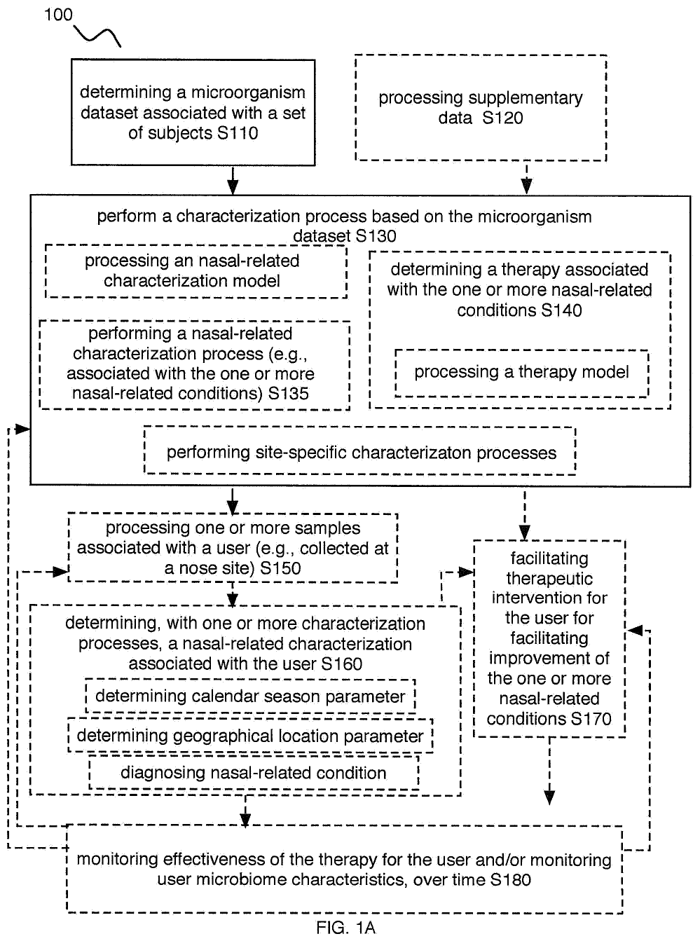

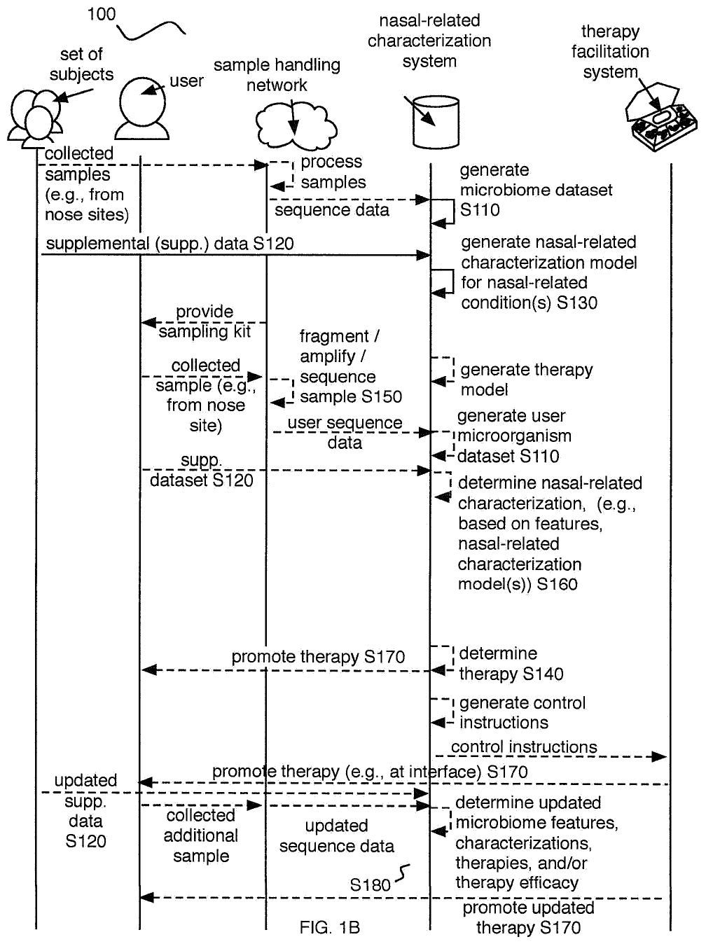

As shown in FIG. 1A-1B, embodiments of a method 100 (e.g., for nasal-related characterization associated with microorganisms, etc.) can include: determining a microorganism dataset (e.g., microorganism sequence dataset; microbiome composition diversity dataset such as based upon a microorganism sequence dataset; microbiome functional diversity dataset such as based upon a microorganism sequence dataset, etc.) associated with a set of users (e.g., determining the microorganism dataset based on samples from a set of subjects) S110; and/or performing a characterization process (e.g., pre-processing, feature determination, feature processing, nasal-related characterization model processing, etc.), based on the microorganism dataset (e.g., based on microbiome composition features and/or microbiome functional features derived from the microorganism dataset and associated with, the one or more nasal-related conditions; etc.) S130, where performing the characterization process can additionally or alternatively include performing a nasal-related characterization process (e.g., for calendar season prediction associated with samples; for one or more nasal-related conditions; etc.) S135, and/or determining one or more therapies (e.g., determining therapies for preventing, ameliorating, reducing the risk of, and/or otherwise improving the one or more nasal-related conditions, etc.) S140.

Embodiments of the method 100 can additionally or alternatively include one or more of: processing supplementary data (e.g., for use in determining one or more nasal-related characterizations; etc.) S120; processing one or more biological samples associated with a user (e.g., subject, human, animal, patient; etc.) S150; determining, with one or more characterization processes, a nasal-related characterization associated with the user, based on a user microorganism dataset (e.g., user microorganism sequence dataset; user microbiome composition dataset; user microbiome function dataset; user microbiome features derived from the user microorganism dataset, where the user microbiome features can correspond to feature values for the microbiome features determined from one or more characterization processes; etc.) associated with a biological sample of the user (e.g., derived from sample processing and/or sequencing of the biological sample, etc.) S160; facilitating therapeutic intervention (e.g., providing a therapy to the user) for one or more nasal-related conditions for the user (e.g., based upon the nasal-related characterization and/or a therapy model; for facilitating improvement of the one or more nasal-related conditions; etc.) S170; monitoring effectiveness of one or more therapies and/or monitoring other suitable components (e.g., microbiome characteristics, etc.) for the user (e.g., based upon processing a series of biological samples from the user), over time (e.g., such as to assess user microbiome characteristics such as user microbiome composition features and/or functional features associated with the therapy, for the user over time, etc.) S180; and/or any other suitable processes.

In a specific example, the method 100 (e.g., for nasal-related characterization associated with microorganisms, etc.) can include determining a microorganism sequence dataset associated with a set of subjects (e.g., including subjects associated with geographic diversity, climate type diversity, age diversity, demographic diversity, etc.; including subjects with one or more nasal-related conditions; including subjects without the nasal-related conditions, where samples and/or data associated with such subjects can act as a control; etc.), based on microorganism nucleic acids from samples collected from nose sites (and/or other suitable body sites) of the set of subjects; determining a set of microbiome features including at least one of a set of microbiome composition features and a set of microbiome functional features, based on the microorganism sequence dataset (e.g., extracted based on applying analytical techniques with the microorganism sequence dataset and/or supplementary data associated with the samples; etc.); generating a nasal-related characterization model (e.g., for predicting calendar season, geographic location, climate status and/or other suitable parameters for one or more samples; etc.) based on the set of microbiome composition features and supplementary data associated with the set of subjects (e.g., supplementary data including one or more of calendar seasons, geographic locations, climate statuses, ages, sampling times, nasal-related condition data, and/or other data associated with the samples and/or subjects; etc.); and/or determining one or more nasal-related characterizations (e.g., determining a calendar season associated with a user sample, such as in relation to sampling time; determining a characterization of one or more nasal-related conditions for the user; etc.) associated with a user based on the nasal-related characterization model and a user sample collected at a nose site (and/or other suitable body sites) of the user.

In a specific example, the method 100 (e.g., for nasal-related characterization associated with microorganisms, etc.) can include collecting a sample from a user (e.g., via sample kit provision and collection, etc.), where the sample is from a nose site (and/or other suitable body sites) of the user and includes microorganism nucleic acids; determining a microorganism dataset associated with the user based on the microorganism nucleic acids of the sample (e.g., based on sample preparation and/or sequencing with the sample, etc.); determining user microbiome features (e.g., including at least one of user microbiome composition features and/or user microbiome functional features, etc.) based on the microorganism dataset; and/or determining one or more nasal-related characterizations (e.g., determining a calendar season associated with a user sample, such as in relation to sampling time; determining a characterization of one or more nasal-related conditions for the user; etc.).

Embodiments of the method 100 and/or system 200 can function to determine one or more nasal-related characterizations (e.g., calendar season predictions for a sample; geographical location predictions for a sample, such as in relation to origin; characterizations for one or more nasal-related conditions; etc.), such as based on a nose microbiome of a user at a given point in time (e.g., accounting for geographic and temporal variation in a nose microbiome, in relation to predicting origin and/or seasonality for a collected sample; etc.).

In specific examples, nasal-related characterizations can be based on microbiome composition features (e.g., relative abundance features) associated with at least one of Abiotrophia, Achromobacter, Acinetobacter, Actinobacillus, Actinomyces, Aggregatibacter, Alistipes, Alloprevotella, Anaerococcus, Anaerostipes, Anoxybacillus, Aquabacterium, Arthrobacter, Atopobium, Bacillus, Bacteroides, Bergeyella, Bifidobacterium, Blautia, Bradyrhizobium, Brevibacterium, Brevundimonas, Burkholderia, Campylobacter, Capnocytophaga, Caulobacter, Centipeda, Chryseobacterium, Collinsella, Corynebacterium, Deinococcus, Delftia, Dermabacter, Dialister, Dolosigranulum, Dorea, Enterobacter, Faecalibacterium, Finegoldia, Flavobacterium, Fusicatenibacter, Fusobacterium, Gemella, Granulicatella, Haemophilus, Herbaspirillum, Hydrogenophilus, Klebsiella, Kluyvera, Kocuria, Lactobacillus, Lactococcus, Lautropia, Leptotrichia, Malassezia, Megasphaera, Meiothermus, Methylobacterium, Micrococcus, Moraxella, Mycobacterium, Negativicoccus, Neisseria, Novosphingobium, Ochrobactrum, Pantoea, Parabacteroides, Parvimonas, Pelomonas, Peptoniphilus, Peptostreptococcus, Phyllobacterium, Porphyromonas, Prevotella, Propionibacterium, Pseudobutyrivibrio, Pseudomonas, Ralstonia, Rhizobium, Roseburia, Rothia, Sarcina, Shinella, Sphingomonas, Staphylococcus, Stenotrophomonas, Streptococcus, Veillonella, Parasutterella, Rhodopseudomonas, Xanthomonas, Mesorhizobium, Facklamia, Kingella, Rhodobacter, Lysinibacillus, Dermacoccus, Cardiobacterium, and/or other suitable taxa (e.g., informative of geographic location parameters; calendar season and/or other sampling time parameters; nasal-related conditions; etc.).

Additionally or alternatively, embodiments of the method 100 and/or system 200 can function to identify microbiome features, supplemental features (e.g., derived from supplemental data, etc.), and/or other suitable data associated with (e.g., positive correlated with, negatively correlated with, etc.) one or more nasal-related characterizations (e.g., associated with nasal-relation conditions; etc.), such as for use as biomarkers (e.g., for diagnostic processes, for treatment processes, etc), for use in sample identification and/or tracking, and/or other suitable purposes. In examples, associations between microbiome features and environmental factors (e.g., calendar season, other sampling time parameters; geographic location; living conditions; climate type; etc.) can be identified, applied, and/or otherwise use (e.g., for predicting environmental factors associated with new samples, etc.). In examples, nasal-related characterization can be associated with at least one or more of microbiome composition (e.g., microbiome composition diversity, etc.), microbiome function (e.g., microbiome functional diversity, etc.), and/or other suitable microbiome-related aspects. In an example, microorganism features (e.g., describing composition, function, and/or diversity of recognizable patterns, such as in relation to relative abundance of microorganisms that are present in a user's microbiome, such as for subjects exhibiting one or more nasal-related conditions; etc.) and/or microorganism datasets (e.g., from which microbiome features can be derived, etc.) can be used for characterizations (e.g., diagnoses, risk assessments, etc.), therapeutic intervention facilitation, monitoring, and/or other suitable purposes, such as by using bioinformatics pipelines, analytical techniques, and/or other suitable approaches described herein. Additionally or alternatively, embodiments of the method 100 and/or system 200 can function to perform cross-condition analyses for a plurality of nasal-related conditions (e.g., performing characterization processes for a plurality of nasal-related conditions, such as determining correlation, covariance, comorbidity, and/or other suitable relationships between different nasal-related conditions, etc.), such as in the context of characterizing (e.g., diagnosing; providing information related to; etc.) and/or treating a user.

Additionally or alternatively, embodiments can function to facilitate therapeutic intervention (e.g., therapy selection; therapy promotion and/or provision; therapy monitoring; therapy evaluation; etc.) for one or more nasal-related conditions, such as through promotion of associated therapies (e.g., in relation to specific body sites such as a nose site and/or other suitable body sites including any one or more of gut site, skin site, mouth site, genital site, other collection sites; therapies determined by therapy models; etc.). Additionally or alternatively, embodiments can function to generate models (e.g., nasal-related characterization models such as for sample metadata prediction, user characteristic prediction, and/or phenotypic prediction; therapy models such as for therapy determination; machine learning models such as for feature processing; etc.), such as models that can be used to characterize and/or diagnose users based on their microbiome (e.g., user microbiome features; as a clinical diagnostic; as a companion diagnostic, etc.), and/or that can be used to select and/or provide therapies for subjects in relation to one or more nasal-related conditions. Additionally or alternatively, embodiments can perform any suitable functionality described herein.

As such, data from populations of users (e.g., populations of subjects associated with one or more nasal-related conditions; positively or negatively correlated with one or more nasal-related conditions; etc.) can be used to characterize subsequent users, such as for indicating microorganism-related states of health and/or areas of improvement, and/or to facilitate therapeutic intervention (e.g., promoting one or more therapies; facilitating modulation of the composition and/or functional diversity of a user's microbiorne toward one or more of a set of desired equilibrium states, such as states correlated with improved health states associated with one or more nasal-related conditions; etc.), such as in relation to one or more nasal-related conditions. Variations of the method 100 can further facilitate selection, monitoring (e.g., efficacy monitoring, etc.) and/or adjusting of therapies provided to a user, such as through collection and analysis (e.g., with nasal-related characterization models) of additional samples from a user over time (e.g., over multiple calendar seasons; throughout the course of a therapy regimen, through the extent of a user's experiences with nasal-related conditions; etc.), across body sites (e.g., across sample collection sites of a user, such as collection sites corresponding to a particular body site type such as a nose site, gut site, mouth site, skin site, genital site; etc.), in addition or alternative to processing supplementary data over time, such as for one or more nasal-related conditions. However, data from populations, subgroups, individuals, and/or other suitable entities can be used by any suitable portions of embodiments of the method 100 and/or system 200 for any suitable purpose.

Embodiments of the method 100 and/or system 200 can preferably determine and/or promote (e.g., provide; present; notify regarding; etc.) characterizations and/or therapies for one or more nasal-related conditions, and/or any suitable portions of embodiments of the method 100 and/or system 200 can be performed in relation to nasal-related conditions.

Nasal-related conditions can include one or more of: sinus infections, nasal polyps, Hay fever, nasal septum deviation, allergies, rhinitis (e.g., chronic atrophic rhinitis, nonallergic rhinitis, etc.), rhinorrhea, chronic rhinosinusitis, bacterial nasal infections, nosebleeds, post-nasal drip, recurrent respiratory papillomatosis, laryngeal papillomatosis, runny nose, sinus tumors, stuffy nose, nasal congestion, carcinoma (e.g., sinonasal undifferentiated carcinoma, etc.), fungal sinusitis, ansomia, choanal atresia, primary ciliary dyskinesia, inverted papilloma, objects in the nose, nasopharynx cancer, aspergillosis, respiratory tract infections, infectious diseases, and/or any suitable conditions associated with the nose and/or nasal passages.

Additionally or alternatively, nasal-related conditions can include one or more of: diseases, symptoms, causes (e.g., triggers; etc.), associated severity, behaviors (e.g., physical activity behavior; alcohol consumption; smoking behaviors; stress-related characteristics; other psychological characteristics; sickness; social behaviors; caffeine consumption; alcohol consumption; sleep habits; other habits; diet-related behaviors such as fiber intake, fruit intake, vegetable intake; meditation and/or other relaxation behaviors; lifestyle conditions associated with nasal-related conditions; lifestyle conditions informative of, correlated with, indicative of, facilitative of, and/or otherwise associated with diagnosis and/or therapeutic intervention for nasal-related conditions; behaviors affecting and/or otherwise associated with the nose and/or nasal-related conditions; etc.), environmental factors (e.g., calendar season, other sampling time, geographic location, climate type, etc.), demographic-related characteristics (e.g., age, weight, race, gender, etc.), phenotypes (e.g., phenotypes measurable for a human, animal, plant, fungi body; phenotypes associated with nose and/or other related aspects, etc.), and/or any other suitable aspects associated with nasal-related conditions. In an example, one or more nasal-related conditions can interfere with normal physical, mental, social and/or emotional function.

Embodiments of the method 100 and/or system 200 can be implemented for a single user, such as in relation to applying one or more sample handling processes and/or characterization processes for processing one or more biological samples (e.g., collected across one or more collection sites, etc.) from the user, for nasal-related characterization, facilitating therapeutic intervention, and/or for any other suitable purpose. Additionally or alternatively, embodiments can be implemented for a population of subjects (e.g., including the user, excluding the user), where the population of subjects can include subjects similar to and/or dissimilar to any other subjects for any suitable type of characteristics (e.g., in relation to nasal-related conditions, demographic characteristics, behaviors, microbiome composition and/or function, etc.); implemented for a subgroup of users (e.g., sharing characteristics, such as characteristics affecting nasal-related characterization and/or therapy determination; etc.); implemented for plants, animals, microorganisms, and/or any other suitable entities. Thus, information derived from a set of subjects (e.g., population of subjects, set of subjects, subgroup of users, etc.) can be used to provide additional insight for subsequent users. In a variation, an aggregate set of biological samples is preferably associated with and processed for a wide variety of subjects, such as including subjects of one or more of: different demographic characteristics (e.g., genders, ages, marital statuses, ethnicities, nationalities, socioeconomic statuses, sexual orientations, etc.), different nasal-related conditions (e.g., health and disease states; different genetic dispositions; etc.), different living situations (e.g., living alone, living with pets, living with a significant other, living with children, etc.), different dietary habits (e.g., omnivorous, vegetarian, vegan, sugar consumption, acid consumption, caffeine consumption, etc.), different behavioral tendencies (e.g., levels of physical activity, drug use, alcohol use, etc.), different levels of mobility (e.g., related to distance traveled within a given time period), and/or any other suitable characteristic (e.g., characteristics influencing, correlated with, and/or otherwise associated with microbiome composition and/or function, etc.). In examples, as the number of subjects increases, the predictive power of processes implemented in portions of embodiments of the method 100 and/or system 200 can increase, such as in relation to characterizing subsequent users (e.g., with varying characteristics, etc.) based upon their microbiomes (e.g., in relation to different collection sites for samples for the users, etc.). However, portions of embodiments of the method 100 and/or system 200 can be performed and/or configured in any suitable manner for any suitable entity or entities.

In variations, portions of embodiments of the method 100 can be repeatedly performed in any suitable order and/or any suitable components of embodiments of the system 200 can be repeatedly applied, such as to improve any suitable portions of embodiments of the method 100 and/or any suitable components of embodiments of the system 200. In an example, portions of embodiments of the method 100 can be repeatedly performed to enable refining of one or more microorganism databases (e.g., improving taxonomic databases through identifying new markers associated with different taxa and/or conditions, such as by collecting and analyzing additional samples, such as samples collected from subjects over time, the course of one or more nasal-related conditions, and/or therapeutic interventions; etc.); refining of the characterization process (e.g., through updating reference abundances used to compare against user relative abundances of targets, such as for identifying clinically relevant results; through generation and updating of characterization models; through increasing the number of conditions that can be characterized using a single biological sample; etc.); the therapy process (e.g., through monitoring and modulating microbiome composition with therapies over time such as through iteratively performing characterization processes over time, such as where the therapies can be selected based on characterization results possessing sensitivity, specificity, precision, and negative predictive value; etc.), and/or other suitable processes.

Data described herein (e.g., microbiome features, microorganism datasets, models, nasal-related characterizations, supplementary data, notifications, etc.) can be associated with any suitable temporal indicators (e.g., seconds, minutes, hours, days, weeks, months, calendar seasons, years, etc.) including one or more: temporal indicators indicating when the data was collected (e.g., temporal indicators indicating when a sample was collected; sampling time; etc.), determined, transmitted, received, and/or otherwise processed; temporal indicators providing context to content described by the data (e.g., temporal indicators associated with nasal-related characterizations, such as where the nasal-related characterization describes the nasal-related conditions, sample metadata, user characteristics, and/or user microbiome status at a particular time; etc.); changes in temporal indicators (e.g., changes in nasal-related characterizations and/or nose microbiome over time; changes such as in response to receiving a therapy; latency between sample collection, sample analysis, provision of a nasal-related characterization or therapy to a user, and/or other suitable portions of embodiments of the method 100; etc.); and/or any other suitable indicators related to time.

Additionally or alternatively, parameters, metrics, inputs, outputs, and/or other suitable data can be associated with value types including: scores (e.g., nasal-related condition propensity scores; feature relevance scores; correlation scores, covariance scores, microbiome diversity scores, severity scores; etc.), individual values (e.g., individual nasal-related condition scores, such as condition propensity scores, for different collection sites, etc.), aggregate values, (e.g., overall scores based on individual microorganism-related scores for different collection sites, etc.), binary values (e.g., presence or absence of a microbiome feature; presence or absence of a nasal-related condition; etc.), relative values (e.g., relative taxonomic group abundance, relative microbiome function abundance, relative feature abundance, etc.), classifications (e.g., nasal-related condition classifications and/or diagnoses for users; feature classifications; behavior classifications; demographic characteristic classifications; etc.), confidence levels (e.g., associated with microorganism sequence datasets; with microbiome diversity scores; with other nasal-related characterizations; with other outputs; etc.), identifiers, values along a spectrum, and/or any other suitable types of values. Any suitable types of data described herein can be used as inputs (e.g., for different analytical techniques, models, and/or other suitable components described herein), generated as outputs (e.g., of different analytical techniques, models, etc.), and/or manipulated in any suitable manner for any suitable components associated with the method 100 and/or system 200.

One or more instances and/or portions of embodiments of the method 100 and/or processes described herein can be performed asynchronously (e.g., sequentially), concurrently (e.g., parallel data processing; concurrent cross-condition analysis; multiplex sample processing, such as multiplex amplification of microorganism nucleic acid fragments corresponding to target sequences associated with environmental factors and/or nasal-related conditions; performing sample processing and analysis for substantially concurrently evaluating a panel of nasal-related conditions; computationally determining microorganism datasets, microbiome features, and/or characterizing nasal-related conditions in parallel for a plurality of users; such as concurrently on different threads for parallel computing to improve system processing ability; etc.), in temporal relation (e.g., substantially concurrently with, in response to, serially, prior to, subsequent to, etc.) to a trigger event (e.g., performance of a portion of the method 100), and/or in any other suitable order at any suitable time and frequency by and/or using one or more instances of the system 200, components, and/or entities described herein. In an example, the method 100 can include generating a microorganism dataset based on processing microorganism nucleic acids of one or more biological samples with a next generation sequencing system (e.g., after library preparation including amplification with a bridge amplification substrate; etc.) (and/or other suitable sequencing system), and determining microbiome composition features and/or microbiome functional features at computing devices operable to communicate with the next generation sequencing platform. However, the method 100 and/or system 200 can be configured in any suitable manner.

### 2. EXAMPLES

Microbiome analysis can enable accurate and/or efficient nasal-related characterization (e.g., of a user microbiome, of a user sample, of a user, etc.) and/or therapy provision (e.g, according to portions of embodiments of the method 100, etc.) for nasal-related conditions caused by, correlated with, and/or otherwise associated with microorganisms. Specific examples of the technology can overcome several challenges faced by conventional approaches. First, conventional approaches can require patients to visit one or more care providers to receive a characterization and/or a therapy recommendation, such as for a nasal-related condition, which can amount to inefficiencies and/or health-risks associated with the amount of time elapsed before diagnosis and/or treatment, with inconsistency in healthcare quality, and/or with other aspects of care provider visitation. Second, conventional genetic sequencing and analysis technologies for human genome sequencing can be incompatible and/or inefficient when applied, to the microbiome (e.g., where the human microbiome can include over 10 times more microbial cells than human cells; where viable analytical techniques and the means of leveraging the analytical techniques can differ; where optimal sample processing techniques can differ, such as for reducing amplification bias; where different approaches to nasal-related characterizations can be employed; where the types of conditions and correlations can differ; where causes of the associated conditions and/or viable therapies for the associated conditions can differ; where sequence reference databases can differ; where the microbiome can vary across different body regions of the user such as at different collection sites; where the microbiome, such as the nose microbiome, can vary across geography, climate, calendar season, sampling time, living conditions, other environmental factors, behaviors, etc.). Third, the onset of sequencing technologies (e.g., next-generation sequencing, associated technologies, etc.) has given rise to technological issues (e.g., data processing and analysis issues for the plethora of generated sequence data; issues with processing a plurality of biological samples in a multiplex manner; information display issues; therapy prediction issues; therapy provision issues, etc.) that would not exist but for the unprecedented advances in speed and data generation associated with sequencing genetic material. Specific examples of the method 100 and/or system 200 can confer technologically-rooted solutions to at least the challenges described above.

First, specific examples of the technology can transform entities (e.g., users, biological samples, therapy facilitation systems including, medical devices, etc.) into different states or things. For example, the technology can transform a biological sample into components able to be sequenced and analyzed to generate microorganism dataset and/or microbiome features usable for characterizing users in relation to one or more nasal-related conditions (e.g., such as through use of next-generation sequencing systems, multiplex amplification operations; etc.). In another example, the technology can identify, discourage and/or promote (e.g., present, recommend, provide, administer, etc.), therapies (e.g., personalized therapies based on a nasal-related characterization; etc.) and/or otherwise facilitate therapeutic intervention (e.g., facilitating modification of a user's microbiome composition, microbiome functionality, etc.), which can prevent and/or ameliorate one or more nasal-related conditions, such as thereby transforming the microbiome and/or health of the patient (e.g., improving a health state associated with a nasal-related condition; etc.), such as applying one or more microbiome features (e.g., applying correlations, relationships, and/or other suitable associations between microbiome features and one or more nasal-related conditions; etc.). In another example, the technology can transform microbiome composition and/or function at one or more different body sites of a user (e.g., one or more different collection sites; etc.), such as targeting and/or transforming microorganisms associated with the nose (and/or other suitable sites, such as gut, skin, mouth, and/or genitals, etc.) microbiome (e.g., by facilitating therapeutic intervention in relation to one or more site-specific therapies; etc.). In another example, the technology can control therapy facilitation systems (e.g., dietary systems; automated medication dispensers; behavior modification systems; diagnostic systems; disease therapy facilitation systems; etc.) to promote therapies (e.g., by generating control instructions for the therapy facilitation system to execute; etc.), thereby transforming the therapy facilitation system.

Second, specific examples of the technology can confer improvements in computer-related technology (e.g., improving computational efficiency in storing, retrieving, and/or processing microorganism-related data for nasal-related conditions; computational processing associated with biological sample processing, etc.) such as by facilitating computer performance of functions not previously performable. For example, the technology can apply a set of analytical techniques in a non-generic manner to non-generic microorganism datasets and/or microbiome features (e.g., that are recently able to be generated and/or are viable due to advances in sample processing techniques and/or sequencing technology, etc.) for improving nasal-related characterizations and/or facilitating therapeutic intervention for nasal-related conditions.

Third, specific examples of the technology can confer improvements in processing speed, nasal-related characterization, accuracy, microbiome-related therapy determination and promotion, and/or other suitable aspects, such as in relation to nasal-related conditions. For example, the technology can leverage non-generic microorganism datasets to determine, select, and/or otherwise process microbiome features of particular relevance to one or more types of nasal-related characterizations, such as in relation to environmental factor prediction and/or nasal-related conditions (e.g., processed microbiome features relevant to a nasal-related condition; cross-condition microbiome features with relevance to a plurality of nasal-related conditions, etc.), which can facilitate improvements in accuracy (e.g., by using the most relevant microbiome features; by leveraging tailored analytical techniques; etc.), processing speed (e.g., by selecting a subset of relevant microbiome features; by performing dimensionality reduction techniques; by leveraging tailored analytical techniques; etc.), and/or other computational improvements (e.g., in relation to phenotypic prediction, such as indications of the nasal-related conditions, etc.), other suitable characterizations, therapeutic intervention facilitation, and/or other suitable purposes. In a specific example, the technology can apply feature-selection rules (e.g., microbiome feature-selection rules for composition, function; for supplemental features extracted from supplementary datasets; etc.) to select an optimized subset of features (e.g., microbiome functional features relevant to one or more nasal-related conditions; microbiome composition diversity features such as reference relative abundance features indicative of healthy, presence, absence, and/or other suitable ranges of taxonomic groups associated with nasal-related conditions; user relative abundance features that can be compared to reference relative abundance features correlated with nasal-related conditions and/or therapy responses; etc.) out of a vast potential pool of features (e.g., extractable from the plethora of microbiome data such as sequence data; identifiable by univariate statistical tests; etc.) for generating, applying, and/or otherwise facilitating characterization and/or therapies (e.g., through models, etc.). The potential size of microbiomes (e.g., human microbiomes, animal microbiomes, etc.) can translate into a plethora of data, giving rise to questions of how to process and analyze the vast array of data to generate actionable microbiome insights in relation to nasal-related characterizations. However, the feature-selection rules and/or other suitable computer-implementable rules can enable one or more of: shorter generation and execution times (e.g., for generating and/or applying models; for determining nasal-related characterizations and/or associated therapies; etc.); optimized sample processing techniques (e.g., improving transformation of microorganism nucleic acids from biological samples through using primer types, other biomolecules, and/or other sample processing components identified through computational analysis of taxonomic groups, sequences, and/or other suitable data associated with nasal-related conditions, such as while optimizing for improving specificity, reducing amplification bias, and/or other suitable parameters; etc.); model simplification facilitating efficient interpretation of results; reduction in overfitting; network effects associated with generating, storing, and applying nasal-related characterizations for a plurality of users over time in relation to nasal-related conditions (e.g., through collecting and processing an increasing amount of microbiome-related data associated with an increasing number of users to improve predictive power of the nasal-related characterizations and/or therapy determinations; etc.); improvements in data storage and retrieval (e.g., storing and/or retrieving nasal-related characterization models; storing specific models such as in association with different users and/or sets of users, with different nasal-related conditions; storing microorganism datasets in association with user accounts; storing therapy monitoring data in association with one or more therapies and/or users receiving the therapies; storing features, nasal-related characterizations, and/or other suitable data in association with a user, set of users, and/or other entities to improve delivery of personalized characterizations and/or treatments for the nasal-related conditions, etc.), and/or other suitable improvements to technological areas.

Fourth, specific examples of the technology can amount to an inventive distribution of functionality across components including a sample handling system, a nasal-related characterization system, and a plurality of users, where the sample handling system can handle substantially concurrent processing of biological samples (e.g., in a multiplex manner) from the plurality of users, which can be leveraged by the nasal-related characterization system in generating personalized characterizations, and/or therapies (e.g., customized to the user's microbiome such as in relation to the user's dietary behavior, probiotics-associated behavior, medical history, demographic characteristics, other behaviors, preferences, etc.) for nasal related conditions.

Fifth, specific examples of the technology can improve the technical fields of at least genomics, microbiology, microbiome-related computation, diagnostics, therapeutics, microbiome-related digital medicine, digital medicine generally, modeling, and/or other relevant fields. In an example, the technology can model and/or characterize different nasal-related conditions, such as through computational identification of relevant microorganism features (e.g., which can act as biomarkers to be used in diagnoses, facilitating therapeutic intervention, etc.) for nasal-related conditions. In another example, the technology can perform cross condition analysis to identify and evaluate cross-condition microbiome features associated with (e.g., shared across, correlated across, etc.) a plurality of a nasal-related conditions (e.g., diseases, phenotypes, etc.). Such identification and characterization of microbiome features can facilitate improved health care practices (e.g., at the population and individual level, such as by facilitating diagnosis and therapeutic intervention, etc.), by reducing risk and prevalence of comorbid and/or multi-morbid nasal-related conditions (e.g., which can be associated with environmental factors, and thereby associated with the microbiome, etc.). In specific examples, the technology can apply unconventional processes (e.g., sample processing processes; computational analysis processes; etc.), such as to confer improvements in technical fields.

Sixth, the technology can leverage specialized computing devices (e.g., devices associated with the sample handling system, such as next-generation sequencing systems; nasal-related characterization systems; therapy facilitation systems; etc.) in performing suitable portions associated with embodiments of the method 100 and/or system 200.

Specific examples of the technology can, however, provide any suitable improvements in the context of using non-generalized components and/or suitable components of embodiments of the system 200 for nasal-related characterization, microbiome modulation, and/or for performing suitable portions of embodiments of the method 100.

### 3.1 DETERMINING A MICROORGANISM DATASET

Embodiments of the method 100 can include Block S110, which can include determining a microorganism dataset (e.g., microorganism sequence dataset, microbiome composition diversity dataset such as based upon a microorganism sequence dataset, microbiome functional diversity dataset such as based upon a microorganism sequence dataset, etc.) associated with a set of users Silo. Block S110 can function to process samples (e.g., biological samples; non-biological samples; an aggregate set of samples associated with a population of subjects, a subpopulation of subjects, a subgroup of subjects sharing a demographic characteristic and/or other suitable characteristics; a user sample; etc.), in order to determine compositional, functional, pharmacogenomics, and/or other suitable aspects associated with the corresponding microbiomes, such as in relation to one or more nasal-related conditions.

Compositional and/or functional aspects can include one or more of aspects at the microorganism level (and/or other suitable granularity), including parameters related to distribution of microorganisms across different groups of kingdoms, phyla, classes, orders, families, genera, species, subspecies, strains, and/or any other suitable infraspecies taxon (e.g, as measured in total abundance of each group, relative abundance of each group, total number of groups represented, etc.). Compositional and/or functional aspects can also be represented in terms of operational taxonomic units (OTUs). Compositional and/or functional aspects can additionally or alternatively include compositional aspects at the genetic level (e.g., regions determined by multilocus sequence typing, 16S sequences, 18S sequences, ITS sequences, other genetic markers, other phylogenetic markers, etc.). Compositional and functional aspects can include the presence or absence or the quantity of genes associated with specific functions (e.g. enzyme activities, transport functions, immune activities, etc.). Outputs of Block S110 can thus be used to facilitate determination of microbiome features (e.g., generation of a microorganism sequence dataset usable for identifying microbiome features; etc.) for the characterization process of Block S130 and/or other suitable portions of embodiments of the method 100 (e.g., where Block S110 can lead to outputs of microbiome composition datasets, microbiome functional datasets, and/or other suitable microorganism datasets from which microbiome features can be extracted, etc.), where the features can be microorganism-based (e.g., presence of a genus of bacteria), genetic-based (e.g., based upon representation of specific genetic regions and/or sequences), functional-based (e.g., presence of a specific catalytic activity), and/or any other suitable microbiome features.

In a variation, Block S110 can include assessment and/or processing based upon phylogenetic markers (e.g., for generating microorganism datasets, etc.) derived from bacteria and/or archaea in relation to gene families associated with one or more of: ribosomal protein S2, ribosomal protein S3, ribosomal protein S5, ribosomal protein S7, ribosomal protein S8, ribosomal protein S9, ribosomal protein S10, ribosomal protein S11, ribosomal protein S12/S23, ribosomal protein S13, ribosomal protein S15P/S13e, ribosomal protein S17, ribosomal protein S19, ribosomal protein L1, ribosomal protein L2, ribosomal protein L3, ribosomal protein L4/L1e, ribosomal protein L5, ribosomal protein L6, ribosomal protein L10, ribosomal protein L11, ribosomal protein L14b/L23e, ribosomal protein L15, ribosomal protein L16/L10E, ribosomal protein L18P/L5E, ribosomal protein L22, ribosomal protein L24, ribosomal protein L25/L23, ribosomal protein L29, translation elongation factor EF-2, translation initiation factor IF-2, metalloendopeptidase, ffh signal recognition particle protein, phenylalanyl-tRNA synthetase beta subunit, phenylalanyl-tRNA synthetase alpha subunit, tRNA pseudouridine synthase B, Porphobilinogen deaminase, ribosomal protein L13, phosphoribosylformylglycinamidine cyclo-ligase, and ribonuclease HII. Additionally or alternatively, markers can include target sequences (e.g., sequences associated with a microorganism taxonomic group; sequences associated with functional aspects; sequences correlated with nasal-related conditions; sequences indicative of user responsiveness to different therapies; sequences that are invariant across a population and/or any suitable set of subjects, such as to facilitate multiplex amplification using a primer type sharing a primer sequence; conserved sequences; sequences including mutations, polymorphisms; nucleotide sequences; amino acid sequences; etc.), proteins (e.g., serum proteins, antibodies, etc.), peptides, carbohydrates, lipids, other nucleic acids, whole cells, metabolites, natural products, genetic predisposition biomarkers, diagnostic biomarkers, prognostic biomarkers, predictive biomarkers, other molecular biomarkers, gene expression markers, imaging biomarkers, and/or other suitable markers. However, markers can include any other suitable marker(s) associated with microbiome composition, microbiome functionality, and/or nasal-related conditions.

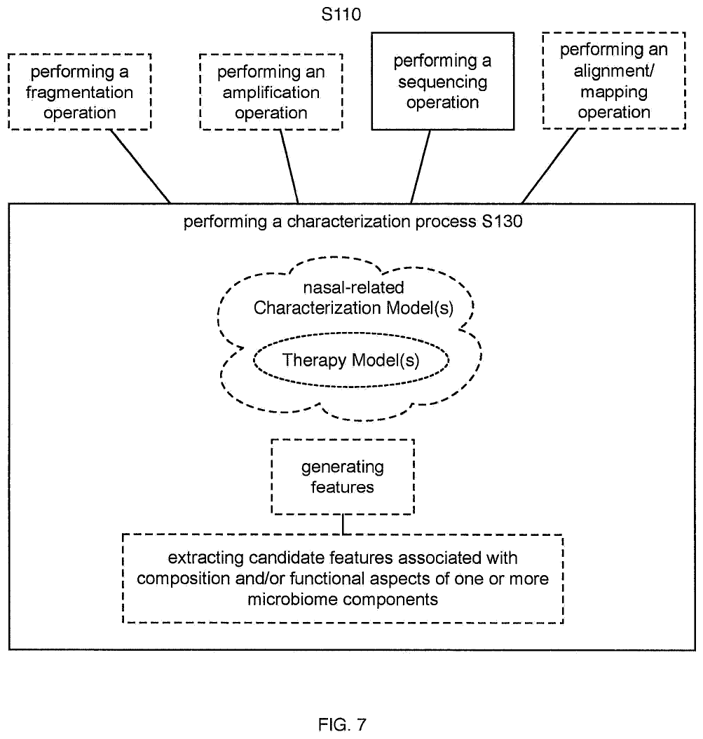

Characterizing the microbiome composition and/or functional aspects for each of the aggregate set of biological samples thus preferably includes a combination of sample processing techniques (e.g., as shown in FIG. 7; wet laboratory techniques; etc.), including, but not limited to, amplicon sequencing (e.g., 16S, 18S, ITS), UMis, 3 step PCR, CRISPR, use of primers, and/or computational techniques (e.g., utilizing tools of bioinformatics), to quantitatively and/or qualitatively characterize the microbiome and functional aspects associated with each biological sample from a subject or population of subjects.

In variations, sample processing in Block S110 can include any one or more of: lysing a biological sample, disrupting membranes in cells of a biological sample, separation of undesired elements (e.g., RNA, proteins) from the biological sample, purification of nucleic acids (e.g., DNA) in a biological sample, amplification of nucleic acids from the biological sample, further purification of amplified nucleic acids of the biological sample, and sequencing of amplified nucleic acids of the biological sample. In an example, Block S110 can include: collecting biological samples from a set of users (e.g., biological samples collected by the user with a sampling kit including a sample container, etc.), where the biological samples include microorganism nucleic acids associated with the nasal-related condition (e.g., microorganism nucleic acids including target sequences correlated with a nasal-related condition; etc.). In another example, Block S110 can include providing a set of sampling kits to a set of users, each sampling kit of the set of sampling kits including a sample container (e.g., including, pre-processing reagents, such as lysing reagents; etc.) operable to receive a biological sample from a user of the set of users.

In variations, lysing a biological sample and/or disrupting membranes in cells of a biological sample preferably includes physical methods (e.g., bead beating, nitrogen decompression, homogenization, sonication), which omit certain reagents that produce bias in representation of certain bacterial groups upon sequencing. Additionally or alternatively, lysing or disrupting in Block S110 can involve chemical methods (e.g., using a detergent, using a solvent, using a surfactant, etc.). Additionally or alternatively, lysing or disrupting in Block S110 can involve biological methods. In variations, separation of undesired elements can include removal of RNA using RNases and/or removal of proteins using proteases. In variations, purification of nucleic acids can include one or more of: precipitation of nucleic acids from the biological samples (e.g., using alcohol-based precipitation methods), liquid-liquid based purification techniques (e.g., phenol-chloroform extraction), chromatography-based purification techniques (e.g., column adsorption), purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.), and any other suitable purification techniques.

In variations, amplification of purified nucleic acids can include one or more of: polymerase chain reaction (PCR)-based techniques (e.g., solid-phase PCR, RT-PCR, qPCR, multiplex PCR, touchdown PCR, nanoPCR, nested PCR, hot start PCR, etc.), helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and any other suitable amplification technique. In amplification of purified nucleic acids, the primers used are preferably selected to prevent or minimize amplification bias, as well as configured to amplify nucleic acid regions/sequences (e.g., of the 16S region, the 18S region, the ITS region, etc.) that are informative taxonomically, phylogenetically, for diagnostics, for formulations (e.g., for probiotic formulations), and/or for any other suitable purpose. Thus, universal primers (e.g., a F27-R338 primer set for 16S RNA, a F515-R806 primer set for 16S RNA, etc.) configured to avoid amplification bias can be used in amplification. Additionally or alternatively include incorporated barcode sequences and/or UMIs specific to biological samples, to users, to nasal-related conditions, to taxa, to target sequences, and/or to any other suitable components, which can facilitate a post-sequencing identification process (e.g., for mapping sequence reads to microbiome composition and/or microbiome function aspects; etc.). In a specific example, applying primers can include amplifying 16S genes (e.g., genes coding for 16S rRNA) with universal V4 primers (e.g., 515F: GTGCCAGCMGCCGCGGTAA and 806R: GGACTACHVGGGTWTCTAAT), other suitable primers associated with variable (e.g., semi-conserved hypervariable regions, etc.) regions (e.g., V1-V8 regions), and/or any other suitable portions of RNA genes. Primers used in variations of Block S110 can additionally or alternatively include adaptor regions configured to cooperate with sequencing techniques involving complementary adaptors (e.g., Illumina Sequencing). Additionally or alternatively, Block S110 can implement any other step configured to facilitate processing (e.g., using a Nextera kit). In a specific example, performing amplification and/or sample processing operations can be in a multiplex manner (e.g., for a single biological sample, for a plurality of biological samples across multiple users; etc.). In another specific example, performing amplification can include normalization steps to balance libraries and detect all amplicons in a mixture independent of the amount of starting material, such as 3 step PCR, bead based normalization, and/or other suitable techniques.

In variations, sequencing of purified nucleic acids can include methods involving targeted amplicon sequencing, implementing techniques including one or more of: sequencing-by-synthesis techniques (e.g., Illumina sequencing), capillary sequencing techniques (e.g., Sanger sequencing), pyrosequencing techniques, and nanopore sequencing techniques (e.g., using an Oxford Nanopore technique).

In a specific example, amplification and sequencing of nucleic acids from biological samples of the set of biological samples includes: solid-phase PCR involving bridge amplification of DNA fragments of the biological samples on a substrate with oligo adapters, where amplification involves primers having a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, UMIs, a sequence for targeting a specific target region (e.g., 16S region, 18S region, ITS region), a reverse index sequence (e.g., corresponding to an Illumina reverse index for MiSeq/HiSeq platforms), and a reverse barcode sequence. In the specific example, sequencing can include Illumina sequencing (e.g., with a HiSeq platform, with a MiSeq platform, with a NextSeq platform, etc.) using a sequencing-by-synthesis technique. In another specific example, the method 100 can include: identifying one or more primer types compatible with one or more genetic targets associated with one or more nasal-related conditions (e.g., a biomarker of the one or more nasal-related conditions; positively correlated with; negatively correlated with; causative of; etc.); determining a microorganism dataset (e.g., microorganism sequence dataset; such as with a next-generation sequencing system; etc.) for one or more users (e.g., set of subjects) based on the one or more primer types (e.g., based on primers corresponding to the one or more primer types, and on the microorganism nucleic acids included in collected biological samples, etc.), such as through fragmenting the microorganism nucleic acids, and/or performing a singleplex amplification process and/or a multiplex amplification process for the fragmented microorganism nucleic acids based on the one or more identified primer types (e.g., primers corresponding to the primer types, etc.) compatible with the one or more genetic targets associated with the nasal-related condition; and/or promoting (e.g., providing), based on a nasal-related characterization derived from a microorganism dataset, a therapy for the user condition (e.g., for the nasal-related condition; enabling selective modulation of a microbiome of the user in relation to at least one of a population size of a desired taxon and a desired microbiome function, etc.). In a specific example, where determining the microorganism dataset can include generating amplified microorganism nucleic acids through at least one of a singleplex amplification process and a multiplex amplification process for the microorganism nucleic acids; and determining, with a next-generation sequencing system, the microorganism dataset based on the amplified microorganism nucleic acids. In an example, determining one or more microorganism sequence datasets can be based on sequencing the microorganism nucleic acids (e.g., from collected samples, etc.) with a next-generation sequencing system.

In examples, the biological samples can correspond to a one or more collection sites including at least one of a gut collection site (e.g., corresponding to a body site type of a gut site), a skin collection site (e.g., corresponding to a body site type of a skin site), a nose collection site (e.g., corresponding to a body site type of a nose site), a mouth collection site (e.g., corresponding to a body site type of a mouth site), and a genitals collection site (e.g., corresponding to a body site type of a genital site). In a specific example, determining a microorganism dataset (e.g., microorganism sequence dataset, etc) can include identifying a first primer type compatible with a first genetic target associated with one or more nasal-related conditions and a first collection site of the set of collection sites; identifying a second primer type compatible with a second genetic target associated'with the one or more nasal-related conditions and a second collection site of the set of collection sites; and generating the microorganism dataset for the set of subjects based on the microorganism nucleic acids, the first primers corresponding to the first primer type, and second primers corresponding to the second primer type.

In variations, primers (e.g., of a primer type corresponding to a primer sequence; etc.) used in Block S110 and/or other suitable portions of embodiments of the method 100 can include primers associated with protein genes (e.g., coding for conserved protein gene sequences across a plurality of taxa, such as to enable multiplex amplification for a plurality of targets and/or taxa; etc.). Primers can additionally or alternatively be associated with nasal-related conditions (e.g., primers compatible with genetic targets including microorganism sequence biomarkers for microorganisms correlated with nasal-related conditions; etc.), microbiome composition features (e.g., identified primers compatible with a genetic target corresponding to microbiome composition features associated with a group of taxa correlated with a nasal-related condition; genetic sequences from which relative abundance features are derived etc.), functional diversity features, supplementary features, and/or other suitable features and/or data. Primers (and/or other suitable molecules, markers, and/or biological material described herein) can possess any suitable size (e.g., sequence length, number of base pairs, conserved sequence length, variable region length, etc.). Additionally or alternatively, any suitable number of primers can be used in sample processing for performing characterizations (e.g., nasal-related characterizations; etc.), improving sample processing (e.g., through reducing amplification bias, etc.), and/or for any suitable purposes. The primers can be associated with any suitable number of targets, sequences, taxa, conditions, and/or other suitable aspects. Primers used in Block S110 and/or other suitable portions of embodiments of the method 100 can be selected through processes described in Block S110 (e.g., primer selection based on parameters used in generating the taxonomic database) and/or any other suitable portions of embodiments of the method 100. Additionally or alternatively, primers (and/or processes associated with primers) can include and/or be analogous to that described in U.S. application No. 14/919,614, filed 21 Oct. 2015, which is herein incorporated in its entirety by this reference. However, identification and/or usage of primers can be configured in any suitable manner.

Some variations of sample processing can include further purification of amplified nucleic acids (e.g., PCR products) prior to sequencing, which functions to remove excess amplification elements (e.g., primers, dNTPs, enzymes, salts, etc.). In examples, additional purification can be facilitated using any one or more of: purification kits, buffers, alcohols, pH indicators, chaotropic salts, nucleic acid binding filters, centrifugation, and/or any other suitable purification technique.

In variations, computational processing in Block S110 can include any one or more of: identification of microbiome-derived sequences (e.g., as opposed to subject sequences and contaminants), alignment and mapping of microbiome-derived sequences (e.g., alignment of fragmented sequences using one or more of single-ended alignment, ungapped alignment, gapped alignment, pairing), and generating features associated with (e.g., derived from) compositional and/or functional aspects of the microbiome associated with a biological sample.

Identification of microbiome-derived sequences can include mapping of sequence data from sample processing to a subject reference genome (e.g., provided by the Genome Reference Consortium), in order to remove subject genome-derived sequences. Unidentified sequences remaining after mapping of sequence data to the subject reference genome can then be further clustered into operational taxonomic units (OTUs) based upon sequence similarity and/or reference-based approaches (e.g., using VAMPS, using MG-RAST, using QIIME databases), aligned (e.g., using a genome hashing approach, using a Needleman-Wunsch algorithm, using a Smith-Waterman algorithm), and mapped to reference bacterial genomes (e.g., provided by the National Center for Biotechnology Information), using an alignment algorithm (e.g., Basic Local Alignment Search Tool, FPGA accelerated alignment tool, BWT-indexing with BWA, BWT-indexing with SOAP, BWT-indexing with Bowtie, etc.). Mapping of unidentified sequences can additionally or alternatively include mapping to reference archaeal genomes, viral genomes and/or eukaryotic genomes. Furthermore, mapping of taxa can be performed in relation to existing databases, and/or in relation to custom-generated databases.

Any suitable processes described in Block 8120 can be performed in a multiplex manner for any suitable number of biological samples. In an example, Block 8120 can include barcoding a plurality of samples with forward and reverse indexes (e.g., unique combinations), sequencing the plurality of samples in a multiplex manner; and, after sequencing, demultiplexing the samples corresponding to different users (e.g., with a BCL2FASTQ algorithm, etc.). Additionally or alternatively, any number of instances of portions of Block S110 can be performed at any suitable time and frequency. However, processing biological samples, determining microorganism datasets, and/or other associated aspects can be performed in any suitable manner analogous to that described in U.S. application Ser. No. 16/047,840 filed 27 Jul. 2018, which is herein incorporated in its entirety by this reference.

In examples, microorganism datasets can be determined for facilitating one or more nasal-related characterization processes (e.g., in relation to Blocks S130, S135, S160, and/or suitable portions of embodiments of the method 100, etc.). In a specific example, the method 100 can include collecting a set of samples from a set of subjects (e.g., self-sampled by the set of subjects; sampled at nose sites of the set of subjects; etc.); subsetting the set of subjects based on one or more environmental factors (e.g., seasons and climates, according to sampling time such as sampling dates; etc.); extracting DNA from the set of samples (e.g., using magnetic beads; etc.); amplifying targets from the DNA (e.g., V4 region of the 16S rRNA region; using barcoded primers; etc.); sequencing the amplified library (e.g., paired-end sequencing; using one or more next-generation sequencing systems; etc.); applying one or more analytical techniques (e.g., in relation to performing a characterization process; etc.), such as aligning both ends to a previously curated amplicon dataset after chimeras removal, to facilitate determination of a microorganism dataset (e.g., a processed microorganism dataset; etc.) and/or determination of microbiome features (e.g., microbiome composition features; etc.), such as where taxonomic counts, abundance, and/or other suitable data associated with microbiome composition can be analyzed in combination with supplementary data (e.g., sample metadata including calendar season, climate types, other environmental factors, etc.) for nasal-related characterization (e.g., in relation to Block S130, S135, S160, and/or suitable portions of embodiments of the method 100, etc.). In a specific example, climate types can be assigned (e.g., to samples, etc.) using Major Koppen climate classification types based on geographic location of the sample and/or user

However, processing biological samples, generating a microorganism dataset, and/or other associated aspects can be performed in any suitable manner.

### 3.2 PROCESSING SUPPLEMENTARY DATA

Embodiments of the method 100 can additionally or alternatively include Block S120, which can include processing (e.g., receiving, collecting, transforming, determining supplementary features, ranking supplementary features, identifying correlations, etc.) supplementary data (e.g., one or more supplementary datasets, etc.), such as supplementary data associated with (e.g., informative of; describing; indicative of; correlated with; etc.) nasal-related characterization (e.g., supplementary data for use in determining nasal-related characterizations; etc.), one or more nasal-related conditions, one or more users, and/or other suitable entities. Block S120 can function to process data for supplementing microorganism datasets, microbiome features (e.g., in relation to determining nasal-related characterizations and/or facilitating therapeutic intervention, etc.), and/or can function to supplement any suitable portion of the method 100 and/or system 200 (e.g., processing supplementary data for facilitating one or more characterization processes, such as in Block S130; such as for facilitating training, validating, generating, determining, applying and/or otherwise processing nasal-related characterization models, etc.). In an example, supplementary data can include at least one of survey-derived data, user data, site-specific data, and device data (and/or other suitable supplementary data), where an example of method 100 can include determining a set of supplementary features based on the at least one of the survey-derived data, the user data, the site-specific data, and the device data (and/or other suitable supplementary data); and processing (e.g., generating, training, applying, etc.) one or more nasal-related characterization models based on the supplementary features, microbiome features, and/or other suitable data.

Supplementary data can include one or more of: sample metadata (e.g., geographical location parameters, calendar season parameters, climate types, for one or more samples; environmental factors (e.g., described herein); survey-derived data (e.g., data from responses to one or more surveys surveying for sample metadata, user data, one or more nasal-related conditions, for any suitable types of data described herein; etc.); site-specific data (e.g., data informative of a nose site; different collection sites, such as prior biological knowledge indicating correlations between microbiomes at specific collection sites and one or more nasal-related conditions; etc.); nasal-related condition data (e.g., data informative of different nasal-related conditions, such as in relation to microbiome characteristics, therapies, users, etc.); device data (e.g.., sensor data; contextual sensor data associated with nose; wearable device data; medical device data; user device data such as mobile phone application data; web application data; etc.); user data (e.g., age; ethnicity; user medical data current and historical medical data such as historical therapies, historical medical examination data; medical device-derived data; physiological data; data associated with medical tests; social media data; demographic data; family history data; behavior data describing behaviors; environmental factor data describing environmental factors such as living conditions; diet-related data such as data from food establishment check-ins, data from spectrophotometric analysis, user-inputted data, nutrition data associated with probiotic and/or prebiotic food items, types of food consumed, amount of food consumed, caloric data, diet regimen data, and/or other suitable diet-related data; etc.); prior biological knowledge (e.g., informative of nasal-related conditions, microbiome characteristics, associations between microbiome characteristics and nasal-related conditions, etc.); and/or any other suitable type of supplementary data.

In variations, processing supplementary data can include processing survey-derived data, where the survey-derived data can provide sample metadata, condition data (e.g., indicating presence, absence, and/or severity of one or more nasal-related conditions; etc.), physiological data, demographic data, behavior data, environmental factor data (e.g., describing environmental factors, etc.), other types of supplementary data, and/or any other suitable data.

Physiological data can include information related to physiological features (e.g., height, weight, body mass index, body fat percent, body hair level, medical history, etc.). Demographic data can include information related to demographic characteristics (e.g., gender, age, ethnicity, marital status, number of siblings, socioeconomic status, sexual orientation, etc.). Behavioral data can describe behaviors including one or more: health-associated states (e.g., health and disease states), dietary habits (e.g., alcohol consumption, caffeine consumption, omnivorous, vegetarian, vegan, sugar consumption, acid consumption, consumption of wheat, egg, soy, treenut, peanut, shellfish, food preferences, allergy characteristics, consumption and/or avoidance of other food items, etc.), behavioral tendencies (e.g., levels of physical activity, drug use, alcohol use, habit development, etc.), different levels of mobility (e.g., amount of exercise such as low, moderate, and/or extreme physical exercise activity; related to distance traveled within a given time period; indicated by mobility sensors such as motion and/or location sensors; etc.), different levels of sexual activity (e.g., related to numbers of partners and sexual orientation), and any other suitable behavioral data. Survey-derived data can include quantitative data, qualitative data, and/or other suitable types of survey-derived data, such as where qualitative data can be converted to quantitative data (e.g., using scales of severity, mapping of qualitative responses to quantified scores, etc.). Processing survey-derived data can include facilitating collection of survey-derived data, such as by providing one or more surveys to one or more users, subjects, and/or other suitable entities. Surveys can be provided in-person (e.g., in coordination with sample kit provision and/or reception of samples; etc.), electronically (e.g., during account setup; at an application executing at an electronic device of a subject, at a web application and/or website accessible through an Internet connection; etc.), and/or in any other suitable manner.

Additionally or alternatively, processing supplementary data can include processing sensor data (e.g., sensors of nasal-related devices, wearable computing devices, mobile devices; biometric sensors associated with the user, such as biometric sensors of a user smart phone; etc.). Sensor data can include any one or more of: physical activity- and/or physical action-related data (e.g., accelerometer data, gyroscope data, location sensor data such as GPS data, and/or other mobility sensor data from one or more devices such as a mobile device and/or wearable electronic device, etc.), sensor data describing environmental factors (e.g., temperature data, elevation data, climate data, light parameter data, pressure data, air quality data, etc.), biometric sensor data (e.g., blood pressure data; temperature data; pressure data associated with swelling; heart rate sensor data; fingerprint sensor data; optical sensor data such as facial images and/or video; data recorded through sensors of a mobile device; data recorded through a wearable or other peripheral device; etc.), and/or any other suitable data associated with sensors. Additionally or alternatively, sensor data can include data sampled at one or more: optical sensors (e.g., image sensors, light sensors, cameras, etc.), audio sensors (e.g., microphones, etc.), temperature sensors, volatile compound sensors, air quality sensors, weight sensors, humidity sensors, depth sensors, location sensors (GPS receivers; beacons; indoor positioning systems; compasses; etc.), motion sensors (e.g., accelerators, gyroscope, magnetometer, motion sensors integrated with a device worn by a user, etc.), biometric sensors (e.g., heart rate sensors such as for monitoring heart rate; fingerprint sensors; facial recognition sensors; bio-impedance sensors, etc.), pressure sensors, proximity sensors (e.g., for monitoring motion and/or other aspects of third-party objects; etc.), flow sensors, power sensors (e.g., Hall effect sensors), virtual reality-related sensors, augmented reality-related sensors, and/or or any other suitable types of sensors.

Additionally or alternatively, supplementary data can include medical record data and/or clinical data. As such, portions of the supplementary dataset can be derived from one or more electronic health records (EHRs). Additionally or alternatively, supplementary data can include any other suitable diagnostic information (e.g., clinical diagnosis information). Any suitable supplementary data (e.g., in the form of extracted supplementary features, etc.) can be combined with and/or used with microbiome features and/or other suitable data for performing portions of embodiments of the method 100 (e.g., performing characterization processes, etc.) and/or system 200. For example, supplementary data associated with (e.g., derived from, etc.) computed tomography (CT scan), ultrasound, biopsy, blood test, cancer screening exams, urine test (e.g., to detect infection; etc.), diagnostic imaging, other suitable diagnostic procedures associated with nasal-related conditions, survey-related information, and/or any other suitable test can be used to supplement (e.g., for any suitable portions of embodiments of the method 100 and/or system 200).

Additionally or alternatively, supplementary data can include therapy-related data including one or more of: therapy regimens, types of therapies, recommended therapies, therapies used by the user, therapy adherence, and/or other suitable data related to therapies. For example, supplementary data can include user adherence metrics (e.g., medication adherence, probiotic adherence, physical exercise adherence, dietary adherence, etc.) in relation one or more therapies (e.g., a recommended therapy, etc.). However, processing supplementary data can be performed in any suitable manner.

### 3.3 PERFORMING A CHARACTERIZATION PROCESS

Embodiments of the method 100 can include Block S130, which can include, performing a characterization process (e.g., pre-processing; feature generation; feature processing; site-specific characterization, such as characterization specific to one or more particular body sites, such as for samples collected at collection sites corresponding to the body site, such as multi-site characterization for a plurality of body sites; cross-condition analysis for a plurality of nasal-related conditions; model generation; etc.) associated with one or more nasal-related conditions, such as based on a microorganism dataset (e.g., derived in Block S110, etc.) and/or other suitable data (e.g., supplementary dataset; etc.) S130. Block S130 can function to identify, determine, extract, and/or otherwise process features and/or feature combinations that can be used to determine nasal-related characterizations for users or and sets of users, based upon their microbiome composition (e.g., microbiome composition diversity features, etc.), function (e.g., microbiome functional diversity features, etc.), and/or other suitable microbiome features (e.g., such as through the generation and application of a characterization model for determining nasal-related characterizations, etc.).

As such, the characterization process can be used as a diagnostic tool that can characterize a subject (e.g., in terms of behavioral traits, in terms of medical conditions, in terms of demographic characteristics, etc.) based upon their microbiome composition and/or functional features, in relation to one or more of their health condition states (e.g., nasal-related condition states), behavioral traits, medical conditions, demographic characteristics, and/or any other suitable traits. Such characterizations can be used to determine, recommend, and/or provide therapies (e.g., personalized therapies, such as determined by way of a therapy model, etc.), and/or otherwise facilitate therapeutic intervention. Additionally or alternatively, characterization processes can be based on microorganism databases (e.g., including associations between one or more microbiome features and one or more nasal-related conditions; etc.).

Performing a characterization process S130 can include pre-processing microorganism datasets, microbiome features, and/or other suitable data for facilitation of downstream processing (e.g., determining nasal-related characterizations, etc.). In an example, performing a characterization process can include, filtering a microorganism dataset (e.g., filtering a microorganism sequence dataset, such as prior to applying a set of analytical techniques to determine the microbiome features, etc.), by at least one of: a) removing first sample data corresponding to first sample outliers of a set of biological samples (e.g., associated with one or more nasal-related conditions, etc.), such as where the first sample outliers are determined by at least one of principal component analysis, a dimensionality reduction technique, and a multivariate methodology; b) removing second sample data corresponding to second sample outliers of the set of biological samples, where the second sample outliers can determined based on corresponding data quality for the set of microbiome features (e.g., removing samples corresponding to a number of microbiome features with high quality data below a threshold condition, etc.); and c) removing one or more microbiome features from the set of microbiome features based on a sample number for the microbiome feature failing to satisfy a threshold sample number condition, where the sample number corresponds to a number of samples associated with high quality data for the microbiome feature. However, pre-processing can be performed with any suitable analytical techniques in any suitable manner.

In performing the characterization process, Block S130 can use computational methods (e.g., statistical methods, machine learning methods, artificial intelligence methods, bioinformatics methods, etc.) to characterize a subject, as exhibiting features (e.g., where determining user microbiome features can include determining feature values for microbiome features identified by characterization processes as correlated with and/or otherwise associated with one or more nasal-related conditions, etc.) associated with one or more nasal-related conditions (e.g., features characteristic of a set of users with the one or more nasal-related conditions, etc.).

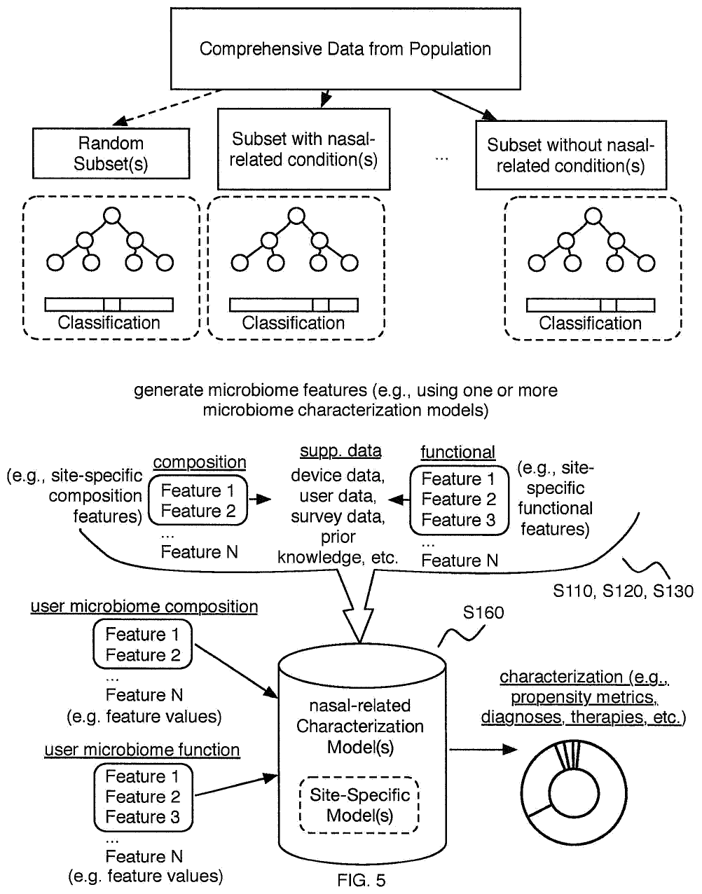

As shown in FIG. 5, performing characterization processes can include determining one or more microbiome features associated with one or more nasal-related characterizations (e.g., identifying microbiome features with greatest relevance to one or more nasal-related conditions; determining user microbiome features, such as presence, absence, and/or values of user microbiome features corresponding to the identified microbiome features associated with the one or more nasal-related conditions, etc.), such as through applying one or more analytical techniques. In an example, determining microbiome features (e.g., microbiome composition features, microbiome functional features, etc.) can applying a set of analytical techniques including at least one of a univariate statistical test, a multivariate statistical test, a dimensionality reduction technique, and an artificial intelligence approach, such as based on a microorganism dataset (e.g., microorganism sequence dataset, etc.), and where the microbiome features can be configured to improve computing system-related functionality associated with the determining of the nasal-related characterization for the user (e.g., in relation to accuracy, reducing error, processing speed, scaling, etc.). In an example, determining microbiome features (e.g., user microbiome features, etc.) can include applying a set of analytical techniques to determine at least one of presence of at least one of a microbiome composition diversity feature and a microbiome functional diversity feature, absence of the at least one of the microbiome composition diversity feature and the microbiome functional diversity feature, a relative abundance feature describing relative abundance of different taxonomic groups associated with the first nasal-related condition, a ratio feature describing a ratio between at least two microbiome features associated with the different taxonomic groups, an interaction feature describing an interaction between the different taxonomic groups, and a phylogenetic distance feature describing phylogenetic distance between the different taxonomic groups, such as based on the microorganism dataset, and where the set of analytical techniques can include at least one of a univariate statistical test, a multivariate statistical test, a dimensionality reduction technique, and an artificial intelligence approach.

In variations, upon identification of represented groups of microorganisms of the microbiome associated with a biological sample, generating features associated with (e.g., derived from) compositional and functional aspects of the microbiome associated with a biological sample can be performed. In a variation, generating features can include generating features based upon multilocus sequence typing (MSLT), in order to identify markers useful for characterization in subsequent blocks of the method 100. Additionally or alternatively, generated features can include generating features that describe the presence or absence of certain taxonomic groups of microorganisms, and/or ratios between exhibited taxonomic groups of microorganisms. Additionally or alternatively, generating features can include generating features describing one or more of: quantities of represented taxonomic groups, networks of represented taxonomic groups, correlations in representation of different taxonomic groups, interactions between different taxonomic groups, products produced by different taxonomic groups, interactions between products produced by different taxonomic groups, ratios between dead and alive microorganisms (e.g., for different represented taxonomic groups, based upon analysis of RNAs), phylogenetic distance (e.g., in terms of Kantorovich-Rubinstein distances, Wasserstein distances etc.), any other suitable taxonomic group-related feature(s), any other suitable genetic or functional aspect(s).

Additionally or alternatively, generating features can include generating features describing relative abundance of different microorganism groups, for instance, using a sparCC approach, using Genome Relative Abundance and Average size (GAAS) approach and/or using a Genome Relative Abundance using Mixture Model theory (GRAMMy) approach that uses sequence-similarity data to perform a maximum likelihood estimation of the relative abundance of one or more groups of microorganisms. Additionally or alternatively, generating features can include generating statistical measures of taxonomic variation, as derived from abundance metrics. Additionally or alternatively, generating features can include generating features associated with (e.g., derived from) relative abundance factors (e.g., in relation to changes in abundance of a taxon, which affects abundance of other taxa). Additionally or alternatively, generating features can include generation of qualitative features describing presence of one or more taxonomic groups, in isolation and/or in combination. Additionally or alternatively, generating features can include generation of features related to genetic markers (e.g., representative 16S, IBS, and/or ITS sequences) characterizing microorganisms of the microbiome associated with a biological sample. Additionally or alternatively, generating features can include generation of features related to functional associations of specific genes and/or organisms having the specific genes. Additionally or alternatively, generating features can include generation of features related to pathogenicity of a taxon and/or products attributed to a taxon. Block S130 can, however, include determination of any other suitable feature(s) derived from sequencing and mapping of nucleic acids of a biological sample. For instance, the feature(s) can be combinatory (e.g. involving pairs, triplets), correlative (e.g., related to correlations between different features), and/or related to changes in features (e.g., temporal changes, changes across sample sites, etc., spatial changes, etc.). However, determining microbiome features can be performed in any suitable manner.

In variations, performing a characterization process can include performing one or more multi-site analyses (e.g., with nasal-related characterization models; generating a multi-site characterization, etc.) associated with a plurality of collection sites, such as performing nasal-related characterizations based on a set of site-specific features including a first subset of site-specific features associated with a first body site, and a second subset of site-specific features associated with a second body site. However, multi-site analyses can be performed in any suitable manner.

In variations, performing a characterization process can include performing one or more cross-condition analyses (e.g., using nasal-related characterization models, etc.) for a plurality of nasal-related conditions. In an example, performing cross-condition analyses can include determining a set of cross-condition features (e.g., as part of determining microbiome features, etc.) associated with a plurality of nasal-related conditions (e.g., a first nasal-related condition and a second nasal-related condition, etc.) based on one or more analytical techniques, where determining a nasal-related characterization can include determining the nasal-related-characterization for a user for the plurality of nasal-related conditions (e.g., first and the second nasal-related conditions, etc.) based on one or more nasal-related characterization models, and where the set of cross-condition features is configured to improve the computing system-related functionality associated with the determining of the nasal-related characterization for the user for the plurality of nasal-related conditions. Performing cross-condition analyses can include determining cross-condition correlation metrics (e.g., correlation and/or covariance between data corresponding to different nasal-related conditions, etc.) and/or other suitable metrics associated with cross-condition analyses. However, performing cross-condition analyses can be performed in any suitable manner.

In a variation, characterization can be based upon features associated with (e.g., derived from) a statistical analysis (e.g., an analysis of probability distributions) of similarities and/or differences between a first group of subjects exhibiting a target state (e.g., a nasal-related condition state) and a second group of subjects not exhibiting the target state (e.g., a “normal” state). In implementing this variation, one or more of a Kolmogorov-Smirnov (KS) test, a permutation test, a Cramér-von Mises test, any other statistical test (e.g., t-test, z-test, chi-squared test, test associated with distributions, etc.), and/or other suitable analytical techniques can be used. In particular, one or more such statistical hypothesis tests can be used to assess a set of features having varying degrees of abundance in a first group of subjects exhibiting a target state (e.g., a sick state) and a second group of subjects not exhibiting the target state (e.g., having a normal state). In more detail, the set of features assessed can be constrained based upon percent abundance and/or any other suitable parameter pertaining to diversity in association with the first group of subjects and the second group of subjects, in order to increase or decrease confidence in the characterization. In a specific implementation of this example, a feature can be derived from a taxon of bacteria that is abundant in a certain percentage of subjects of the first group and subjects of the second group, where a relative abundance of the taxon between the first group of subjects and the second group of subjects can be determined from the KS test, with an indication of significance (e.g., in terms of p-value). Thus, an output of Block Sao can include a normalized relative abundance value (e.g., 25% greater abundance of a taxon in subjects with a nasal-related condition vs. subjects without the nasal-related condition; in sick subjects vs. healthy subjects) with an indication of significance (e.g., a p-value of 0.0013). Variations of feature generation can additionally or alternatively implement or be derived from functional features or metadata features (e.g., non-bacterial markers). Additionally or alternatively, any suitable microbiome features can be derived based on statistical analyses (e.g., applied to a microorganism sequence dataset and/or other suitable microorganism dataset, etc.) including any one or more of: a prediction analysis, multi hypothesis testing, a random forest test, principal component analysis, and/or other suitable analytical techniques.

In performing the characterization process, Block S130 can additionally or alternatively transform input data from at least one of the microbiome composition diversity dataset and microbiome functional diversity dataset into feature vectors that can be tested for efficacy in predicting characterizations of the population of subjects. Data from the supplementary dataset can be used to provide indication of one or more characterizations of a set of characterizations, where the characterization process is trained with a training dataset of candidate features and candidate classifications to identify features and/or feature combinations that have high degrees (or low degrees) of predictive power in accurately predicting a classification. As such, refinement of the characterization process with the training dataset identifies feature sets (e.g., of subject features, of combinations of features) having high correlation with specific classifications of subjects.

In variations, feature vectors (and/or any suitable set of features) effective in predicting classifications of the characterization process can include features related to one or more of: microbiome diversity metrics (e.g., in relation to distribution across taxonomic groups, in relation to distribution across archaeal, bacterial, viral, and/or eukaryotic groups), presence of taxonomic groups in one's microbiome, representation of specific genetic sequences (e.g., 16S sequences) in one's microbiome, relative abundance of taxonomic groups in one's microbiome, microbiome resilience metrics (e.g., in response to a perturbation determined from the supplementary dataset), abundance of genes that encode proteins or RNAs with given functions (enzymes, transporters, proteins from the immune system, hormones, interference RNAs, etc.) and any other suitable features associated with (e.g., derived from) the microbiome diversity dataset and/or the supplementary dataset. In variations, microbiome features can be associated with (e.g., include, correspond to, typify, etc.) at least one of: presence of a microbiome feature from the microbiome features (e.g., user microbiome features, etc.), absence of the microbiome features from the microbiome features, relative abundance of different taxonomic groups associated with the nasal-related condition; a ratio between at least two microbiome features associated with the different taxonomic groups, interactions between the different taxonomic groups, and phylogenetic distance between the different taxonomic groups. In a specific example, microbiome features can include one or more relative abundance characteristics associated with at least one of the microbiome composition diversity features (e.g., relative abundance associated with different taxa, etc.) and the microbiome functional diversity features (e.g., relative abundance of sequences corresponding to different functional features; etc.). Relative abundance characteristics and/or other suitable microbiome features (and/or other suitable data described herein) can be extracted and/or otherwise determined based on: a normalization, a feature vector derived from at least one of linear latent variable analysis and non-linear latent variable analysis, linear regression, non-linear regression, a kernel method, a feature embedding method, a machine learning method, a statistical inference method, and/or other suitable analytical techniques. Additionally or alternatively, combinations of features can be used in a feature vector, where features can be grouped and/or weighted in providing a combined feature as part of a feature set. For example, one feature or feature set can include a weighted composite of the number of represented classes of bacteria in one's microbiome, presence of a specific genus of bacteria in one's microbiome, representation of a specific 16S sequence in one's microbiome, and relative abundance of a first phylum over a second phylum of bacteria. However, the feature vectors can additionally or alternatively be determined in any other suitable manner.

In a variation, the characterization process can be generated and trained according to a random forest predictor (RFP) algorithm that combines bagging (e.g., bootstrap aggregation) and selection of random sets of features from a training dataset to construct a set of decision trees, T, associated with the random sets of features. In using a random forest algorithm, N cases from the set of decision trees are sampled at random with replacement to create a subset of decision trees, and for each node, m prediction features are selected from all of the prediction features for assessment. The prediction feature that provides the best split at the node (e.g., according to an objective function) is used to perform the split (e.g., as a bifurcation at the node, as a trifurcation at the node). By sampling many times from a large dataset, the, strength of the characterization process, in identifying features that are strong in predicting classifications can be increased substantially. In this variation, measures to prevent bias (e.g., sampling bias) and/or account for an amount of bias can be included during processing, such as to increase robustness of the model.

In a variation, Block S130 and/or other portions of embodiments of the method 100 can include applying computer-implemented rules (e.g., models, feature selection rules, etc.) to process population-level data, but can additionally or alternatively include applying computer-implemented rules to process microbiome-related data on a demographic characteristic-specific basis (e.g., subgroups sharing one or more demographic characteristics such as therapy regimens, dietary regimens, physical activity regimens, ethnicity, age, gender, weight, behaviors, etc.), condition-specific basis (e.g., subgroups exhibiting a specific nasal-related condition, a combination of nasal-related conditions, triggers for the nasal-related conditions, associated symptoms, etc.), a sample type-specific basis (e.g., applying different computer-implemented rules to process microbiome data derived from different collection sites; etc.), a user basis (e.g., different computer-implemented rules for different users; etc.) and/or any other suitable basis. As such, Block S130 can include assigning users from the population of users to one or more subgroups; and applying different computer-implemented rules for determining features (e.g., the set of feature types used; the types of characterization models generated from the features; etc.) for the different subgroups. However, applying computer-implemented rules can be performed in any suitable manner.

In another variation, Block S130 can include processing (e.g., generating, training, updating, executing, storing, etc.) one or more nasal-related characterization models (e.g., nasal-related condition models, therapy models, etc) for one or more nasal-related conditions (e.g., for outputting characterizations for users describing user microbiome characteristics in relation to nasal-related conditions; therapy models for outputting therapy determinations for one or more nasal-related conditions; etc.). The characterization models preferably leverage microbiome features as inputs, and preferably output nasal-related characterizations and/or any suitable components thereof; but characterization models can use any suitable inputs to generate any suitable outputs. In an example, Block S130 can include transforming the supplementary data, the microbiome composition diversity features, and the microbiome functional diversity features, other microbiome features, outputs of nasal-related characterization models, and/or other suitable data into one or more characterization models (e.g., training a nasal-related characterization model based on the supplementary data and microbiome features; etc.) for one or more nasal-related conditions. In another example, the method 100 can include: determining a population microorganism sequence dataset (e.g., including microorganism sequence outputs for different users of the population; etc.) for a population of users associated with one or more nasal-related conditions, based on a set of samples from the population of users (e.g., and/or based on one or more primer types associated, with the nasal-related condition; etc.); collecting a supplementary dataset associated with diagnosis of the one or more nasal-related conditions for the population of subjects; and generating the nasal-related characterization model based on the population microorganism sequence dataset and the supplementary dataset. In an example, the method 100 can include determining a set of user microbiome features for the user based on a sample from the user, where the set of user microbiome features is associated with microbiome features associated with a set of subjects (e.g., microbiome features determined to be correlated with one or more nasal-related conditions, based on processing biological samples corresponding to a set of subjects associated with the one or more nasal-related conditions; a set microbiome composition features and the set of microbiome functional features; etc.); determining a nasal-related characterization, including determining a therapy for the user for the one or more nasal-related conditions based on a therapy model and the set of user microbiome features; providing the therapy (e.g., providing a recommendation for the therapy to the user at a computing device associated with the user, etc.) and/or otherwise facilitating therapeutic intervention.

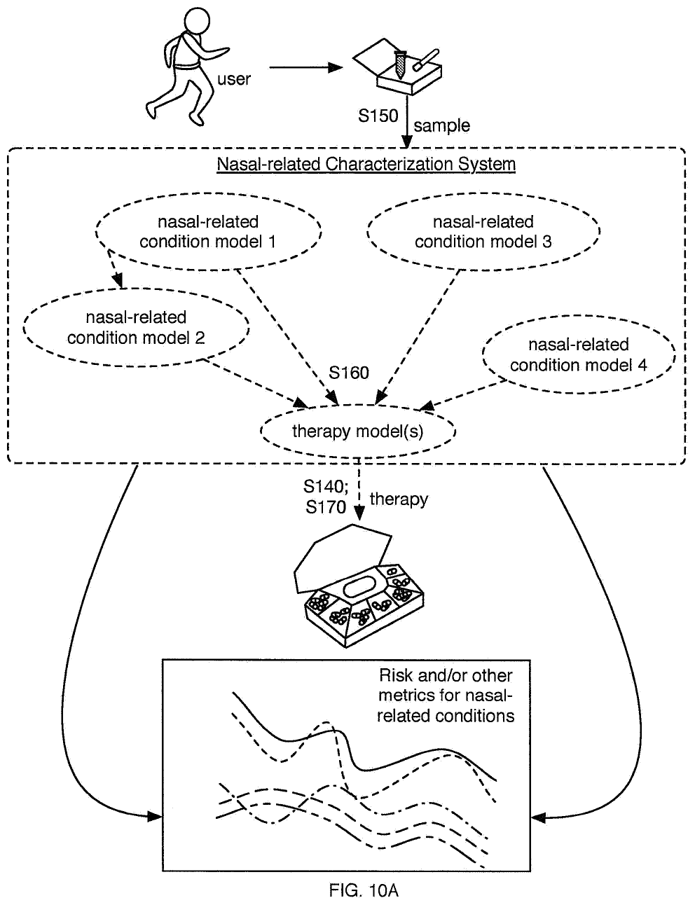

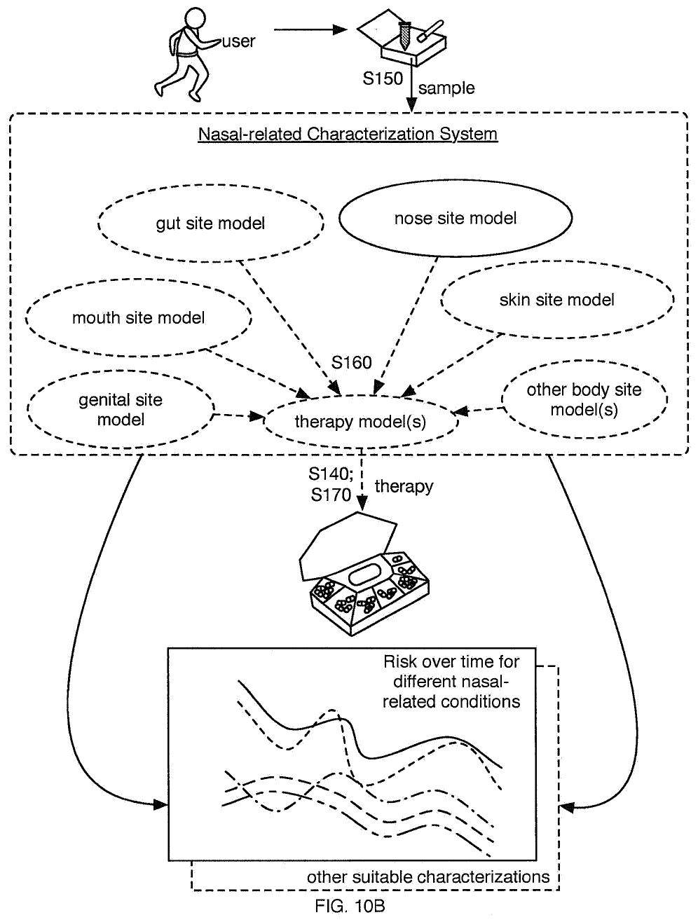

In another variation, as shown in FIG. 10A-10B, different nasal-related characterization models and/or other suitable models (e.g., generated with different algorithms, with different sets of features, with different input and/or output types, applied in different manners such as in relation to time, frequency, component applying the model, etc.) can be generated for different nasal-related conditions, different user demographic characteristics (e.g., based on age, gender, weight, height, ethnicity; etc.), different body sites (e.g., a gut site model, a nose site model, a skin site model, a mouth site model, a genitals site model, etc.), individual users, supplementary data (e.g., models incorporating prior knowledge of microbiome features, nasal-related conditions, and/or other suitable components; features associated with biometric sensor data and/or survey response data vs. models independent of supplementary data, etc.), and/or other suitable criteria. In an specific example, the method 100 can include collecting first site-specific samples associated with a first body site (e.g., a gut site; samples collected by users at body collection sites corresponding to the first body site; one or more suitable body sites; etc.); determining a microorganism dataset based on the site-specific samples; determining first site-specific microbiome features (e.g., site-specific composition features; site-specific functional features; suitable microbiome features described herein in relation to nasal-related conditions; features associated with the first body site; etc.) based on the microorganism dataset; determining a first site-specific nasal-related characterization model (e.g., a gut site-specific nasal-related characterization model; etc.) based on the first site-specific microbiome features; and determining a nasal-related condition for a user for the nasal-related condition based on the first site-specific nasal-related characterization model (e.g., using the first site-specific nasal-related characterization model to process user microbiome features, such as user site-specific microbiome features, derived based on a user sample collected at a body collection site of the user corresponding to the first body site; etc.). In a specific example, the method 100 can include collecting second site-specific samples associated with a second body site (e.g., at least one of a skin site, a genital site, a mouth site, and a nose site; one or more suitable body sites; etc.); determining second site-specific microbiome features (e.g. site-specific composition features; site-specific functional features; features associated with the second body site; etc.);

generating a second site-specific nasal-related characterization model (e.g., associated with the second body site; etc) based on the second site-specific composition features; collecting a user sample from an additional user, the user sample associated with the second body site (e.g., collected by the additional user at a collection site corresponding to the second body site; etc.); and determining an additional nasal-related characterization for the additional user for the nasal-related condition based on the second site-specific nasal-related characterization model (e.g., selecting the second site-specific nasal-related characterization model, from a set of site-specific nasal-related characterization models, to apply based on the association between the user sample and the body site, such as selecting a skin site-specific nasal-related characterization model to apply based on a user sample being collected from a skin collection site of the user; etc.).

In variations, determining nasal-related characterizations and/or any other suitable characterizations can include determining site-specific nasal-related characterizations (e.g., site-specific analyses) including nasal-related characterizations in relation to specific body sites (e.g., gut, healthy gut, skin, nose, mouth, genitals, other suitable body sites, other sample collection sites, etc.), such as through any one or more of: determining a nasal-related characterization based on a nasal-related characterization model derived based on site-specific data (e.g., defining correlations between a nasal-related condition and microbiome features associated with one or more body sites); determining a nasal-related characterization based on a user biological sample collected at one or more body sites, and/or any other suitable site-related processes. In examples, machine learning approaches (e.g., classifiers, deep learning algorithms, SVM, random forest), parameter optimization approaches (e.g., Bayesian Parameter Optimization), validation approaches (e.g., cross validation approaches), statistical tests (e.g., univariate statistical techniques, multivariate statistical techniques, correlation analysis such as canonical correlation analysis, etc.), dimension reduction techniques (e.g., PCA), and/or other suitable analytical techniques(e.g., described herein) can be applied in determining site-related (e.g., body site-related, etc.) characterizations (e.g., using a one or more approaches for one or more sample collection sites, such as for each type of sample collection site, etc.), other suitable characterizations, therapies, and/or any other suitable outputs. In a specific example, performing a characterization process (e.g., determining a nasal-related characterization; determining microbiome features; based on a nasal-related characterization model; etc.) can include applying at least one of: machine learning approaches, parameter optimization approaches, statistical tests, dimension reduction approaches, and/or other suitable approaches (e.g., where microbiome features such as a set of microbiome composition diversity features and/or a set of microbiome functional diversity features can be associated with microorganisms collected at least at one of a gut site, a skin site, a nose site, a mouth site, a genitals site, etc.). In another specific example, characterization processes performed for a plurality of sample collection sites can be used to generate individual characterizations that can be combined to determine an aggregate characterization (e.g., an aggregate microbiome score, such as for one or more conditions described herein, etc.). However, the method 100 can include determining any suitable site-related (e.g., site-specific) outputs, and/or performing any suitable portions of embodiments of the method 100 (e.g., collecting samples, processing samples, determining therapies) with site-specificity and/or other site-relatedness in any suitable manner.

Characterization of the subject(s) can additionally or alternatively implement use of a high false positive test and/or a high false negative test to further analyze sensitivity of the characterization process in supporting analyses generated according to embodiments of the method 100.

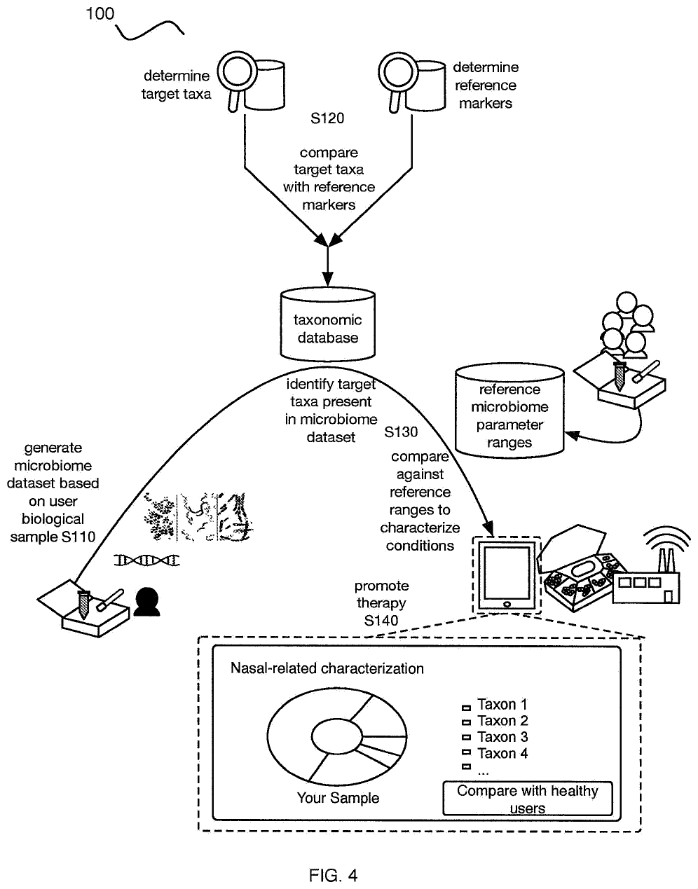

As shown in FIG. 4-5, performing a characterization process can include determining one or more reference microbiome parameter ranges (e.g., a healthy reference relative abundance range, where the range can be associated with a healthy microbiome and/or the absence of one or more conditions; a risk reference relative abundance range associated with the presence of and/or risk of one or more conditions; microorganism composition range for abundance of one or more taxa; phylogenetic diversity of the microorganisms present in the sample; microorganism functional diversity range for functional features associated with one or more taxa; etc.); determining a user microbiome parameter for a user; and/or generating a characterization for the user based on a comparison between the user microbiome parameter and the reference microbiome parameter range (e.g., characterizing a user as possessing an unhealthy microbiome composition in relation to bacterial targets associated with nasal-related characterization based on the user microbiome parameter indicating an abundance outside of the healthy reference ranges for different bacterial targets; etc.) and/or any other suitable operations. Microbiome parameter ranges can have any suitable lower- and upper-limits. Reference microbiome parameter ranges can include ranges representing any suitable confidence intervals (e.g., 99% confidence intervals across a population of users). In an example, reference relative abundance ranges can be calculated for any suitable taxa, such as based on dividing the count of reads corresponding to that taxa by the total number of reads (e.g., total number of clustered and filtered reads); however, reference relative abundance ranges can be calculated in any suitable manner.

In a variation, determining reference microbiome parameter ranges can be performed empirically. For example, Block S130 can include collecting biological samples and supplementary datasets from a population of users. The population of users can include users associated with any suitable state of microbiome composition, microbiome phylogenetic diversity, microbiome functional diversity, conditions, and/or other suitable characteristics, where the supplementary datasets (e.g., digitally administered surveys at an application executing on mobile devices associated with the users) can be informative of the characteristics. In a specific example, the method 100 can include: processing biological samples from a population of healthy users; processing the biological samples (e.g., as in Block S120) to determine microorganism sequences; determining relative abundance of each taxa (e.g., from the target list of taxa) for each user; and generating healthy ranges for each of the taxa based on the relative abundances across the population of healthy users. However, empirically determining reference microbiome parameter ranges can be performed in any suitable manner. In a specific example, the supplementary data can indicate a lack of the at least one nasal-related condition for a subset of subjects from a set of subjects; where determining the set of microbiome features can include determining healthy reference microbiome parameters ranges associated with the subset of subjects, based on the microorganism sequence dataset; and where generating the nasal-related characterization model can include generating the nasal-related characterization model (e.g., a model employing analytical techniques to compare reference microbiome parameter ranges to user microbiome features and/or parameters; etc.) includes based on the supplementary data and the healthy reference microbiome parameters ranges. In a variation, determining reference microbiome parameter ranges can be performed non-empirically, such as based on manually and/or automatically processing condition-related information sources.

In a specific example, performing characterization processes can include determining healthy reference nose microbiome parameter ranges (e.g., health reference nose microbiome relative abundance ranges for different taxa), such as based on analyses of a set of samples, where the samples can be selected based on one or more of: self-reported healthy individuals, no usage of antibiotics six months prior, lack of nasal-related conditions (e.g., lack of infectious diseases over a period of time; etc.), environmental factors (e.g., calendar season, geographical location, etc.), user data, any suitable types of supplementary data, and/or other suitable criteria. However, determining reference microbiome parameter ranges (and/or other suitable microbiome features; etc.) can be performed in any suitable manner.

In variations, determining one or more user microbiome features for a user is preferably based on generated microorganism sequences derived from biological samples of the user (e.g., clustered and filtered reads; etc.). For example, determining a user microbiome feature can include determining a relative abundance for different taxa (e.g., taxa described herein), where such relative abundance features can be compared to reference nose microbiome parameter ranges (e.g., healthy reference nose microbiome parameter ranges; etc.), such as in determining a nasal-related characterization. In a specific example, the method 100 can include: determining reference microbiome parameter ranges from values of microbiome composition features, microbiome phylogenetic diversity features, and/or microbiome functional diversity features (e.g., derived from biological samples of healthy users, etc.); and comparing the user microbiome composition feature values, user microbiome phylogenetic diversity feature values, and/or user microbiome functional, diversity feature values to the reference microbiome parameter ranges to determine nasal-related characterizations for the user (e.g., for conditions positively and/or negatively associated with the reference microbiome parameter ranges; for environmental factors positively and/or negatively associated with the reference microbiome parameter ranges; etc.).

Comparing one or more user microbiome features to one or more reference microbiome features (e.g., parameter ranges, etc.) associated with one or more characteristics (e.g., taxa, conditions, etc.) can include characterizing the user as possessing the characteristic (e.g., a healthy microbiome, etc.) or not possessing the characteristic based on whether the user microbiome parameter values fall inside or outside the reference microbiome parameter ranges.

Additionally or alternatively for Block S130, performing the characterization process can be based on thresholds (e.g., determining risk of a condition based on relative abundances of a set of taxa in relation to a set of thresholds associated with the condition, etc.), weights (e.g., weighting relative abundance of a first taxa more heavily than relative abundance of a second taxa, such as when the first taxa has a greater correlation with the condition of interest, etc.), machine learning models (e.g., a classification model trained on microbiome features and corresponding labels for taxa stored in the taxonomic database; etc.), computer-implemented rules (e.g., feature-engineering rules for extracting microbiome features; model generation rules; user preference rules; microorganism sequence generation rules; sequence alignment rules; etc.), and/or any other suitable aspects. In a specific example, a significance index for each health condition is calculated as the overall statistical association obtained from scientific literature for all members of the microbiome detected that affect the condition; the identified correlations undergo a custom statistical meta-analysis and data transformations to calculate the overall association of the microbiome with the condition, based on the clinical results of the associated microbiome; and the significance index is expressed as a range from 0 to 100 representing the state of the microbiome associated with the health condition.

However, performing one or more characterization processes S130 can be performed in any suitable manner.

### 3.3A NASAL-RELATED CHARACTERIZATION PROCESS

Performing a characterization process S130 can include performing a nasal-related characterization process (e.g., determining a characterization for one or more nasal-related conditions; determining and/or applying one or more nasal-related characterization models; determining nasal-related characterization using any suitable approaches described in relation to Block S130; etc.) S135, such as for one or more samples, users (e.g., for data corresponding to samples from a set of subjects for generating one or more nasal-related characterization models, such as where one or more subjects are associated with one or more environmental factors and/or nasal-related conditions, such as subjects diagnosed with the one or more nasal-related conditions; for a single user for generating a nasal-related characterization for the user, such as through using one or more nasal-related characterization models, such as through applying the one or more nasal-related characterization models to a user microbiome sequence dataset derived from sequencing a sample from the user; etc.), and/or nasal-related conditions.

In variations, performing a nasal-related characterization process can include determining microbiome features associated with one or more nasal-related conditions. In an example, performing a nasal-related characterization process can include applying one or more analytical techniques (e.g., statistical analyses) to identify the sets of microbiome features (e.g., microbiome composition features, microbiome composition diversity features, microbiome functional features, microbiome functional diversity features, etc.) that have the highest correlations (e.g., positive correlations, negative correlations, etc.) with one or more nasal-related conditions (e.g., features associated with a single nasal-related condition, cross-condition features associated with multiple nasal-related conditions and/or other suitable nasal-related conditions, etc.). In a specific example, determining a set of microbiome features (e.g., correlated with and/or otherwise associated with one or more nasal-related conditions; for use in generating one or more nasal-related characterization models; etc) can include applying a set of analytical techniques to determine at least one of presence of at least one of a microbiome composition diversity feature and a microbiome functional diversity feature, absence of the at least one of the microbiome composition diversity feature and the microbiome functional diversity feature, a relative abundance feature describing relative abundance of different taxonomic groups associated with the nasal-related condition, a ratio feature describing a ratio between at least two microbiome features associated with the different taxonomic groups, an interaction feature describing an interaction between the different taxonomic groups, and a phylogenetic distance feature describing phylogenetic distance between the different taxonomic groups, based on the microorganism sequence dataset, and/or where the set of analytical techniques can include at least one of a univariate statistical test, a multivariate statistical test, a dimensionality reduction technique, and an artificial intelligence approach.

In examples, performing a nasal-related characterization process can facilitate therapeutic intervention for one or more nasal-related conditions, such as through facilitating intervention associated with therapies having a positive effect on a state of one or more users in relation to the one or more nasal-related conditions.

In an example, performing a nasal-related characterization process (e.g., determining features with highest correlations to one or more supplementary data types and/or nasal-related conditions; generating a machine learning classifier nasal-related characterization model; etc.) can be based upon applying a random forest approach to train a model with a training dataset derived from a subset of the population of subjects (e.g., subjects and/or samples associated with one or more supplementary; having the one or more nasal-related conditions; subjects not having the one or more nasal-related conditions; etc.), and validated with a validation dataset derived from a subset of the population of subjects.

In variations, performing a nasal-related characterization process can be based on microbiome features and supplementary features (e.g., derived from supplementary data; raw supplementary data; etc.). In an example, microbiome composition features (e.g., taxonomic associated parameters including one or more of counts, abundance, correlation, association index, etc.) can be analyzed in combination with sample metadata (e.g., geographical location, climate type, sampling time, calendar season based on sampling time, etc.) and/or suitable supplementary data (e.g., survey-derived data; etc.). In a specific example, the microbiome composition features and supplementary features can be classified using one or more artificial intelligence approaches, such as both supervised and unsupervised clustering methods (e.g. random forest, support vector machines, k-means clustering, etc.), for generating a nasal-related characterization model (e.g., a high-quality predictor, etc.) that can be used to classify novel samples not present in the dataset, such as for classification in relation to the sample metadata (e.g., calendar season, geographical location, climate type, etc.), other suitable supplementary data types, one or more nasal-related conditions, nose microbiome health, and/or other suitable characteristics.

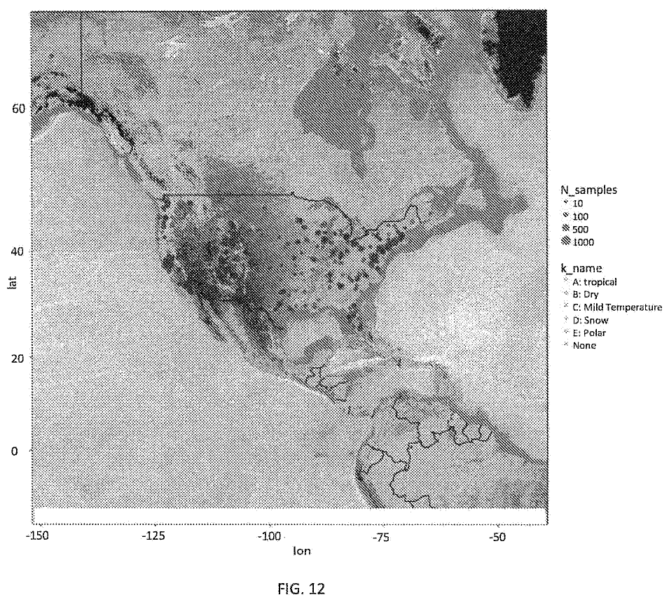

In an example, a random forest approach (and/or other suitable artificial intelligence approaches) can be applied to train one or more nasal-related characterization models to classify nose samples (e.g., a set of nose samples described in FIG. 12, such as in relation to geographic locations, climate types, and number of samples, etc.) by calendar season (and/or other suitable characteristics) based on the nose microbiome (e.g., microbiome features associated with a sample collected from a nose site; etc.). In a specific example, the method 100 can collecting a set of nose site samples from a set of subjects (e.g., sampled at the nose sites of the set of subjects; etc.); tagging the set of nose site samples by calendar season (e.g., based on sampling time determined from survey supplementary data provided by the set of subjects; etc.); determining a microorganism dataset based on the samples; determining microbiome composition features (e.g., genus taxa presence and/or relative abundance in the samples; etc.) based on the microorganism dataset; performing cross validation (e.g., Bayesian optimization; etc.) for generalizing parameters over the dataset (e.g., split into K subsets using different parameters for learning; etc.), such as where the training data is labeled with calendar season (e.g., summer, autumn, winter, spring; where the training data can be split into K subsamples for training temporary models); determining key features (e.g., with greatest correlation and informativeness for the different labels; determining key features per temporary model; microbiome composition features, such as described herein; other microbiome features; supplementary features such as age of subjects or users; etc.); and/or generating one or more nasal-related characterization models (e.g., based on parameters obtained from cross validation, such as eta range, max depth range, number of rounds, subsample rate, bytree range; using feature selection approaches based on the key features; based on all subsamples including data without splitting; based on additional parameters such as a missing values parameter; etc.).

However, determining microbiome features, generating models, and/or other suitable aspects associated with one or more nasal-related related characterizations can be performed in any suitable manner.

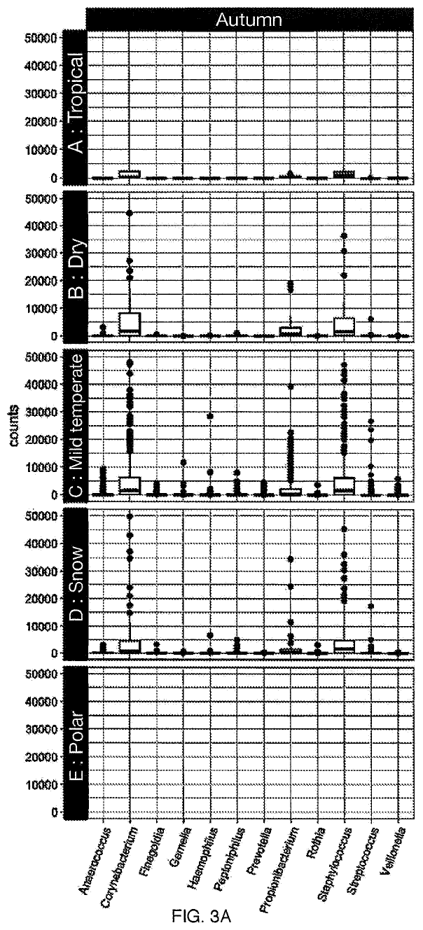

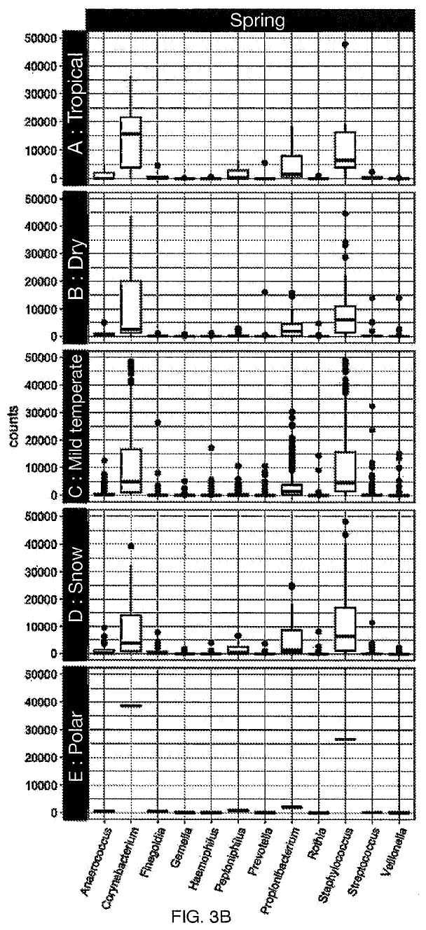

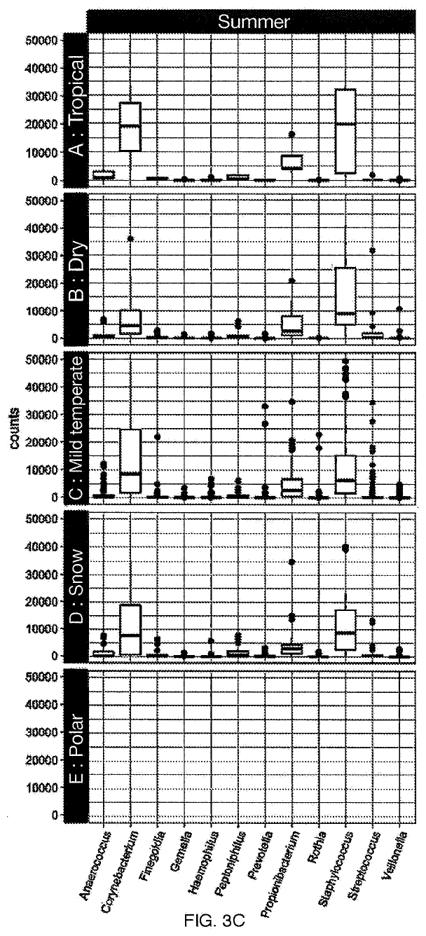

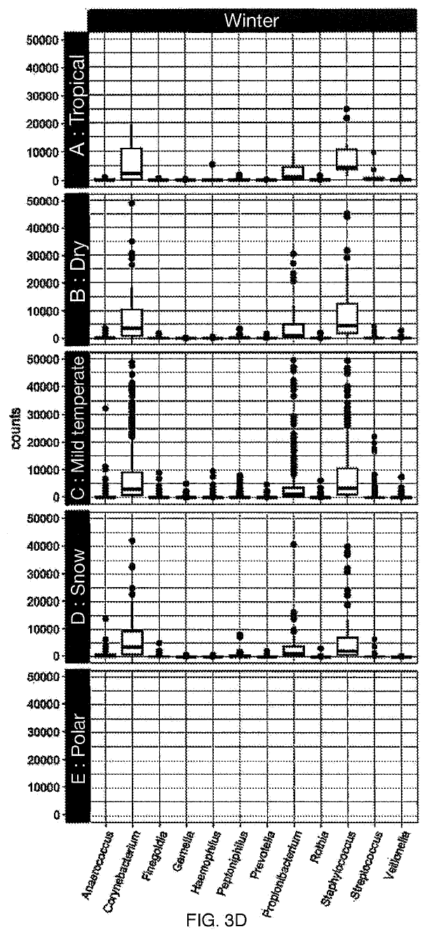

Microbiome features (e.g., microbiome composition features; site-specific composition features associated with one or more body sites; microbiome functional features; site-specific functional features associated with one or more body sites; user microbiome features; etc.) associated with one or more nasal-related characterizations (e.g., positively correlated with; negatively correlated with; useful for diagnosis; etc.) can include features (e.g., microbiome composition features, etc.) associated with any combination of one or more of the following taxa (e.g., features describing abundance of; features describing relative abundance of; features describing functional aspects associated with; features derived from; features describing presence and/or absence of; etc.), such as in relation to one or more body sites (e.g., nose site; where microbiome composition features can include site-specific composition features associated with the one or more body sites, such as where correlations between the composition features and the one or more nasal-related conditions can be specific to the one or more body sites, such as specific to microbiome composition observed at the body site from samples collected at a body collection site corresponding to the body site; etc.): Abiotrophia, Achromobacter, Acinetobacter, Actinobacillus, Actinomyces, Aggregatibacter, Alistipes, Alloprevotella, Anaerococcus, Anaerostipes, Anoxybacillus, Aquabacterium, Arthrobacter, Atopobium, Bacillus, Bacteroides, Bergeyella, Bifidobacterium, Blautia, Bradyrhizobium, Brevibacterium, Brevundimonas, Burkholderia, Campylobacter, Capnocytophaga, Caul obacter, Centipeda, Chryseobacterium, Collinsella, Corynebacterium, Deinococcus, Delftia, Dermabacter, Dialister, Dolosigranulum, Dorea, Enterobacter, Faecalibacteriurn, Finegoldia, Flavobacterium, Fusicatenibacter, Fusobacterium, Gemella, Granulicatella, Haemophilus, Herbaspirillum, Hydrogenophilus, Klebsiella, Kluyvera, Kocuria, Lactobacillus, Lactococcus, Lautropia, Leptotrichia, Malassezia, Megasphaera, Meiothermus, Methylobacterium, Micrococcus, Moraxella, Mycobacterium, Negativicoccus, Neisseria, Novosphingobium, Ochrobactrum, Pantoea, Parabacteroides, Parvimonas, Pelomonas, Peptoniphilus, Peptostreptococcus, Phyllobacterium, Porphyromonas, Prevotella, Propionibacterium, Pseudobutyrivibrio, Pseudomonas, Ralstonia, Rhizobium, Roseburia, Rothia, Sarcina, Shinella, Sphingomonas, Staphylococcus, Stenotrophomonas, Streptococcus, Veillonella, Parasutterella, Rhodopseudomonas, Xanthomonas, Mesorhizobium, Facklamia, Kingella, Rhodobacter, Lysinibacillus, Dermacoccus, Cardiobacterium, and/or other suitable taxa (e.g., described herein). In examples, microbiome features can include any suitable species taxa from the genus Straphyloccus, Corynebacterium, and/or Propionibacterium. In examples, microbiome features can include species (and/or other suitable taxa types), such as Dolosigranulum and/or Moraxella associated with infectious diseases, and/or associated with any suitable nasal-related conditions. In specific examples, as shown in FIG. 3A-3D, microbiome composition features (e.g., key microbiome composition features) including counts (and/or other suitable abundance metrics) for different taxa (e.g., overrepresented taxa; etc.) in relation to climate type and calendar season can be obtained (e.g., from processing a set of nose site samples, such as samples described in FIG. 12), processed (e.g., used for determining features; used in generating and/or applying one or more nasal-related characterization models; etc.), and/or otherwise used, such as for any suitable portions of embodiments of the method 100.

Any suitable microbiome features, supplemental features, and/or other suitable data described herein can be used in processing (e.g., generating, applying, etc.) one or more nasal-related characterization models.

In a specific example, the method 100 can include determining a nasal-related characterization, which can include determining a calendar season parameter (e.g., calendar season prediction of summer, autumn, winter, spring; other suitable seasons; etc.) associated with a user sample collected at the nose site of the user (e.g., a user sample associated with unknown sampling time; etc.), based on a nasal-related characterization model (e.g., generated based on microbiome composition features associated with taxa described herein, and/or on supplementary data; a nasal-related characterization model trained as described in one or more variants described herein; etc.). In a specific example, the nasal-related characterization model can include a calendar season characterization machine learning model, where generating the nasal-related characterization model can include training the calendar season characterization machine learning model based on the set of microbiome composition features and calendar seasons (e.g., determined from supplementary data) associated with the samples collected from the nose sites of the set of subjects, and where determining the calendar season parameter can include determining the calendar season parameter based on the calendar season characterization machine learning model and the user sample collected at the nose site of the user. In a specific example, the supplementary data can include ages of the set of subjects, where generating the nasal-related characterization model can include generating the nasal-related characterization model based on the set of microbiome composition features and the ages of the set of subjects, and where determining the calendar season parameter (e.g., associated with the user sample) can include determining the calendar season parameter based on the nasal-related characterization model, the user sample collected at the nose site of the user, and an age of the user (e.g., where age can be a key supplementary feature for nasal-related characterization, etc.). In a specific example, the supplementary data associated with the set of subjects can include at least one of geographic location, climate type, and sampling time, where generating the nasal-related characterization model can include generating the nasal-related characterization model based on the set of microbiome composition features, the ages of the set of subjects, and the at least one of geographic location, climate type, and sampling time, and where determining the calendar season parameter can include determining the calendar season parameter based on the nasal-related characterization model, the user sample collected at the nose site of the user, the age of the user, and at least one of user geographic location, climate type associated with the user geographic location, and user sampling time associated with, the user sample collected at the nose site of the user. However, any suitable sample time parameters (e.g., calendar season parameters; any suitable time periods such as minutes, hours, days, months, years, etc.) can be predicted, can be associated with nasal-related characterization models, and/or otherwise used.

In a specific example, calendar season-specific characterization models can be processed (e.g., generated, applied, etc.), such as for predictions for a subset of the calendar seasons (e.g., for a subset of summer, autumn, winter, spring; using different sets of features for different subsets; using different sets of features relative a model for a full set of calendar seasons; etc.), such as to improve accuracy, sensitivity, specificity, area under the curve, and/or other suitable metrics associated with models (e.g., relative a predictor model for all calendar seasons). In a specific example, a nasal-related characterization model for spring and winter season predictions (e.g., a binary classification model) can be processed. However, nasal-related characterization models for any subsets of supplementary data types (e.g., a subset of environmental factors, such as a subset of geographical locations, such as a subset of cities, countries, continents, etc.) can be processed in any suitable manner.

In a specific example, determining the nasal-related characterization can include determining (e.g., predicting) a geographic location parameter associated with the user sample (e.g., a user sample associated with unknown geographic location; etc.), such as based on a nasal-related characterization model (e.g., described herein) and a user sample (e.g., collected at a nose site of the user).

In a specific example, the nasal-related characterization model can be associated with one or more nasal-related conditions (e.g., outputs of the model can be informative of one or more nasal-related conditions for one or more users), and determining a nasal-related characterization can include determining the nasal-related characterization for the user for the one or more nasal-related conditions, such as based on the nasal-related characterization model and a user sample collected at the nose site of the user. In a specific example, the method 100 can include facilitating therapeutic intervention (e.g., providing a therapy; providing a therapy recommendation; etc.) to the user for facilitating improvement of the one or more nasal-related condition, based on the one or more nasal-related characterizations.

In a specific example, microbiome features (e.g., microbiome composition features associated with taxa described herein, etc.) can be determined from applying analytical techniques (e.g., random forest approaches, cross validation approaches, etc.) to nose site samples collected from different subsets of subjects, each subset of subjects corresponding to a different calendar season parameter (e.g., summer, autumn, winter, spring) associated with the sampling time, and/or other suitable sampling time parameters.

In a specific example, the method 100 can include determining a nasal-related characterization including characterizing the nose microbiome of one or more users, such as in relation to a panel of taxa (e.g., described herein; relative abundance of the different taxa; for comparison to healthy ranges, to unhealthy ranges, to other users; etc.).

In examples, markers associated with one or more of the plurality of taxa can include 16S rRNA genetic sequences associated with the plurality of taxa. The markers and/or the plurality of taxa can be associated (e.g., positively associated, negatively associated, etc.) with one or more: conditions, pathogens, commensal bacteria, probiotic bacteria, and/or any other marker-associated information, where such associations can be stored in microorganism databases, applied in characterization processes, and/or otherwise processed.

Additionally or alternatively, microbiome features associated with one or more nasal-related characterization can include microbiome functional features (e.g., features describing functions associated with one or more microorganisms, such as microorganisms classified under taxa described herein; features describing functional diversity; features describing presence, absence, abundance, and/or relative abundance; etc.) corresponding to functions from and/or otherwise associated with (e.g., such as in relation to one or more body sites such as nose sites, where microbiome functional features can include site-specific functional features associated with the one or more body sites, such as where correlations between the functional features and the one or more nasal-related characterizations can be specific to the body site, such as specific to microbiome function corresponding to microorganisms observed at the body site from samples collected at a body collection site corresponding to the body site; etc.) one or more of: Clusters of Orthologous Groups (COG) databases (e.g., COG, COG2, etc.), Kyoto Encyclopedia of Genes and Genomes (KEGG) databases (e.g., KEGG2, KEGG3, KEGG4, etc.), and/or any other suitable database available (e.g., databases with microorganism function data, etc.). However, microbiome features can include any suitable microbiome functional features associated with any suitable microorganism function, human function, and/or other suitable functionality.

In variations, site-specific nasal-related characterization models (e.g., for determining nasal-related characterizations based on processing user site-specific microbiome features associated with one or more body sites also associated with the site-specific nasal-related characterization model; etc.) and/or nasal-related characterizations (e.g., associated with a body site, etc.) can be determined based on site-specific microbiome features (e.g., associated with one or more body sites; etc) described herein (e.g., site-specific composition features; site-specific functional features; etc.). In examples, the method 100 can include determining user microbiome features (e.g., for a user for which a nasal-related characterization and/or therapy can be determined and/or promoted; determining feature values for a user for microbiome features determined to be associated with, such as correlated with, the one or more nasal-related conditions; etc.) including site-specific microbiome features associated with one or more body sites.

In specific examples, microbiome composition features (e.g., including site-specific composition features, etc.) described herein, microbiome functional features described herein, and/or other suitable microbiome features can be determined based on one or more microorganism datasets (e.g., microorganism sequence dataset, etc.) determined based on samples (e.g., sequencing of microorganism nucleic acids of the samples, etc.) from a set of subjects associated with one or more environmental factors, other supplementary data types, nasal-related conditions (e.g., a set of subjects including subjects with the nasal-related condition; including subjects without the nasal-related condition, where such samples and/or associated data can act as a control; a population of subjects; etc.), and/or other suitable aspects.

In variations, any suitable combination of microbiome features described herein can be used in prevention, treatment of, and/or suitable facilitation of therapeutic intervention for one or more nasal-related conditions and/or statuses associated with microorganisms, such as for restoring nose microbiota to a healthy cohort (e.g., improving microbiome diversity), such as including modulation of the presence, absence or relative abundance of microorganisms in a nose microbiome and/or other suitable microbiomes associated with suitable body sites (e.g., towards a target microbiome composition and/or functionality associated with users with a healthy microbiome, etc.). However, microbiome features associated with nasal-related conditions can be applied in any suitable manner for prevention, treatment of, and/or suitable facilitation of therapeutic intervention for one or more nasal-related conditions.

In an example, the method 100 can include determining a nasal-related characterization for the user for a first nasal-related condition and a second nasal-related condition based on a first set of composition features (e.g., including at least one or more of the microbiome features described above in relation to the first variation; including any suitable combination of microbiome features; etc.), a first nasal-related characterization model, a second set of composition features (e.g., including at least one or more of the microbiome features described above in relation to the second variation; including any suitable combination of microbiome features; etc.), and a second nasal-related characterization model, where the first nasal-related characterization model is associated with the first nasal-related condition (e.g., where the first nasal-related characterization model determines characterizations for the first nasal-related condition, etc.), and where the second nasal-related characterization model is associated with the second nasal-related condition (e.g., where the second nasal-related characterization model determines characterizations for the second nasal-related condition, etc.). In the example, determining user microbiome features can include determining first user microbiome functional features associated with first functions from at least one of Cluster of Orthologous Groups (COG) database and Kyoto Encyclopedia of Genes and Genomes (KEGG) database, where the first user microbiome functional features are associated with the first nasal-related condition; and determining second user microbiome functional features associated with second functions from at least one of the COG database and the KEGG database, where the second user microbiome functional features are associated with the second nasal-related condition, where determining the nasal related characterization can include determining the nasal-related characterization for the user for the first nasal-related condition and the second nasal-related condition based on the first set of composition features, the first user microbiome functional features, the first nasal-related characterization model, the second set of composition features, the second user microbiome functional features, and the second nasal-related characterization model. Additionally or alternatively, any combinations of microbiome features can be used with any suitable number and types of nasal-related characterization models to determine nasal-related characterization for one or more nasal-related conditions, in any suitable manner.

In examples, the method 100 can include generating one or more nasal-related characterization models based on any suitable combination of microbiome features described above and/or herein (e.g., based on a set of microbiome composition features including features associated with at least one of the taxa described herein; and/or based on microbiome functional features described herein, such as corresponding to functions from databases described herein; etc.). In an example, performing a characterization process for a user can include characterizing a user as having one or more nasal-related conditions, such as based upon detection of, values corresponding to, and/or other aspects related to microbiome features described herein (e.g., microbiome features described above, etc.), and such as in a manner that is an additional (e.g., supplemental to, complementary to, etc.) or alternative to typical approaches of diagnosis, other characterizations (e.g., treatment-related characterizations, etc.), treatment, monitoring, and/or other suitable approaches associated with nasal-related conditions. In variations, the microbiome features can be used for diagnostics, other characterizations, treatment, monitoring, and/or any other suitable purposes and/or approaches associated with nasal-related conditions.

Any suitable taxa, associations, features, and/or other suitable data can be derivable in any suitable manner described in U.S. application Ser. No. 16/047,840 filed 27 Jul. 2018, which is herein incorporated in its entirety by this reference.

However, determining one or more nasal-related characterizations can be performed in any suitable manner.

### 3.3.B DETERMINING A THERAPY

Performing a characterization process S130 (e.g., performing a nasal-related therapy) can include Block S140, which can include determining one or more therapies (e.g., therapies configured to modulate microbiome composition, function, diversity, and/or other suitable aspects, such as for improving one or more aspects associated with nasal-related conditions, such as in users characterized based on one or more characterization processes; etc.). Block S140 can function to identify, select, rank, prioritize, predict, discourage, and/or otherwise determine therapies (e.g., facilitate therapy determination, etc.). For example, Block S140 can include determining one or more of probiotic-based therapies, bacteriophage-based therapies, small molecule-based therapies, and/or other suitable therapies, such as therapies that can shift a subject's microbiome composition, function, diversity, and/or other characteristics (e.g., microbiomes at any suitable sites, etc.) toward a desired state (e.g., equilibrium state, etc.) in promotion of a user's health, for modifying a state of one or more nasal-related conditions, and/or for other suitable purposes.

Therapies (e.g., nasal-related therapies, etc.) can include any one or more of: consumables (e.g., probiotic therapies, prebiotic therapies, medication such as antibiotics, allergy or cold medication, bacteriophage-based therapies, consumables for underlying conditions, small molecule therapies, etc.); device-related therapies (e.g., monitoring devices; sensor-based devices; medical devices; implantable medical devices; etc.); surgical operations; psychological-associated therapies (e.g., cognitive behavioral therapy, anxiety therapy, talking therapy, psychodynamic therapy, action-oriented therapy, rational emotive behavior therapy, interpersonal psychotherapy, relaxation training, deep breathing techniques, progressive muscle relaxation, meditation, etc.); behavior modification therapies (e.g., physical activity recommendations such as increased exercise; dietary recommendations such as reducing sugar intake, increased vegetable intake, increased fish intake, decreased caffeine consumption, decreased alcohol consumption, decreased carbohydrate intake; smoking recommendations such as decreasing tobacco intake; weight-related recommendations; sleep habit recommendations etc.); topical administration therapies (e.g., topical probiotic, prebiotic, and/or antibiotics; bacteriophage-based therapies); environmental factor modification therapies; modification of any other suitable aspects associated with one or more nasal-related conditions; and/or any other suitable therapies (e.g., for improving a health state associated with one or more nasal-related conditions, such as therapies for improving one or more nasal-related conditions, therapies for reducing the risk of one or more nasal-related conditions, etc.). In examples, types of therapies can include any one or more of: probiotic therapies, bacteriophage-based therapies, small molecule-based therapies, cognitive/behavioral therapies, physical rehabilitation therapies, clinical therapies, medication-based therapies, diet-related therapies, and/or any other suitable therapy designed to operate in any other suitable manner in promoting a user's health.

In variations, therapies can include site-specific therapies associated with one or more body sites, such as for facilitating modification of microbiome composition and/or function at one or more different body sites of a user (e.g., one or more different collection sites, etc.), such as targeting and/or transforming microorganisms associated with a nose site and/or other suitable sites including one or more of gut site, skin site, mouth site, and/or genital site; such as by facilitating therapeutic intervention in relation to one or more therapies configured to specifically target one or more user body sites, such as microbiome at one or more of the user body sites; such as for facilitating improvement of one or more nasal-related conditions (e.g., by modifying user microbiome composition and/or function at a particular user body site towards a target microbiome composition and/or function, such as microbiome composition and/or function at a particular body site and associated with a healthy microbiome status and/or lack of the one or more nasal-related condition; etc.). Site-specific therapies can include any one or more of consumables (e.g., targeting a nose site microbiome and/or microbiomes associated with any suitable body sites; etc.); topical therapies (e.g., for modifying a skin microbiome, a nose microbiome, a mouth microbiome, a genitals microbiome, etc.); and/or any other suitable types of therapies. In an example, the method 100 can include collecting a sample associated with a first body site (e.g., including at least one of a nose site, gut site, a skin site, a genital site, a mouth site, and a nose site, etc.) from a user; determining site-specific composition features associated with the first body site; determining a nasal-related characterization for the user for the nasal-related condition based on the site-specific composition features; and facilitating therapeutic intervention in relation to a first site-specific therapy for the user (e.g., providing the first site-specific therapy to the user; etc.) for facilitating improvement of the nasal-related condition, based on the nasal-related characterization, where the first site-specific therapy is associated with the first body site. In an example, the method 100 can include collecting a post-therapy sample from the user after the facilitation of the therapeutic intervention in relation to the first site-specific therapy (e.g., after the providing of the first site-specific therapy; etc.), where the post-therapy sample is associated with a second body site (e.g., including at least one of the nose site, gut site, the skin site, the genital site, the mouth site; etc.); determining a post-therapy nasal-related characterization for the user for the nasal-related condition based on site-specific features associated with the second body site; and facilitating therapeutic intervention in relation to a second site-specific therapy for the user (e.g., providing a second site-specific therapy to the user; etc.) for facilitating improvement of the nasal-related condition, based on the post-therapy nasal-related characterization, where the second site-specific therapy is associated with the second body site.

In a variation, therapies can include one or more bacteriophage-based therapies (e.g., in the form of a consumable, in the form of a topical administration therapy, etc.), where one or more populations (e.g., in terms of colony forming units) of bacteriophages specific to a certain bacteria (or other microorganism) represented in the subject can be used to down-regulate or otherwise eliminate populations of the certain bacteria. As such, bacteriophage-based therapies can be used to reduce the size(s) of the undesired population(s) of bacteria represented in the subject. Additionally or alternatively, bacteriophage-based therapies can be used to increase the relative abundances of bacterial populations not targeted by the bacteriophage(s) used. However, bacteriophage-based therapies can be used to modulate characteristics of microbiomes (e.g., microbiome composition, microbiome function, etc.) in any suitable manner, and/or can be used for any suitable purpose.

In variations, therapies can include one or more probiotic therapies and/or prebiotic therapies associated with any combination of at least one or more of (e.g., including any combination of one or more of, at any suitable amounts and/or concentrations, such as any suitable relative amounts and/or concentrations; etc.) any suitable taxa described herein (e.g., in relation to one or more microbiome composition features associated with one or more nasal-related conditions, etc.), and/or any other suitable microorganisms associated with any suitable taxonomic groups (e.g., microorganisms from taxa described herein, such as in relation to microbiome features; taxa associated with functional features described herein, etc.). For one or more probiotic therapies and/or other suitable therapies, microorganisms associated with a given taxonomic group, and/or any suitable combination of microorganisms can be provided at dosages of 0.1 million to 10 billion CFU, and/or at any suitable amount (e.g., as determined from a therapy model that predicts positive adjustment of a patient's microbiome in response to the therapy; different amounts for different taxa; same or similar amounts for different taxa; etc.). In an example, a subject can be instructed to ingest capsules including the probiotic formulation according to a regimen tailored to one or more of his/her: physiology (e.g., body mass index, weight, height), demographic characteristics (e.g., gender, age), severity of dysbiosis, sensitivity to medications, and any other suitable factor. In examples, probiotic therapies and/or prebiotic therapies can be used to modulate a user microbiome (e.g., in relation to composition, function, etc.) for facilitating improvement of one or more nasal-related conditions. In examples, facilitating therapeutic intervention can include promoting (e.g., recommending, informing a user regarding, providing, administering, facilitating obtainment of, etc.) one or more probiotic therapies and/or prebiotic therapies to a user, such as for facilitating improvement of one or more nasal-related conditions.

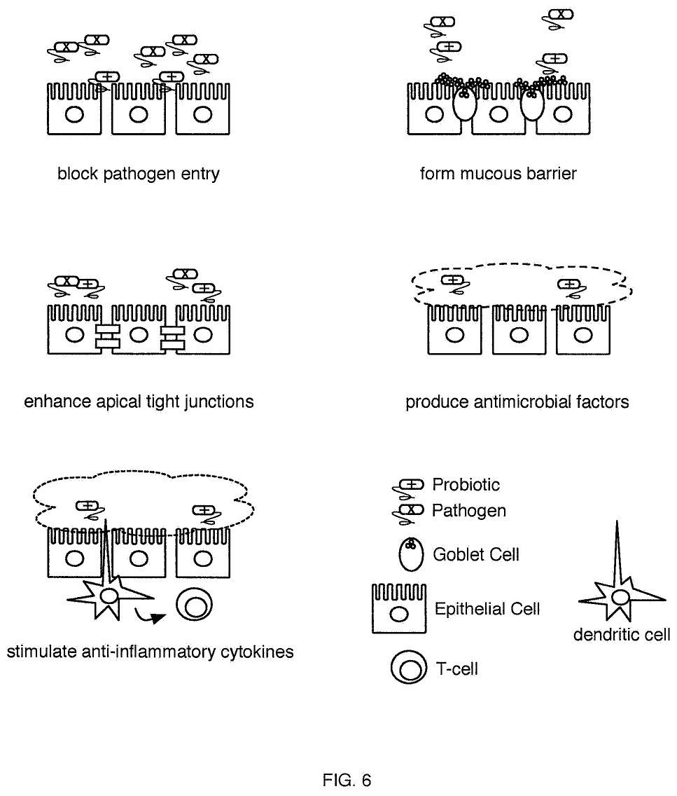

In a specific example of probiotic therapies, as shown in FIG. 6, candidate therapies of the therapy model can perform one or more of: blocking pathogen entry into an epithelial cell by providing a physical barrier (e.g., by way of colonization resistance), inducing formation of a mucous barrier by stimulation of goblet cells, enhance integrity of apical tight junctions between epithelial cells of a subject (e.g., by stimulating up regulation of zona-occludens 1, by preventing tight junction protein redistribution), producing antimicrobial factors, stimulating production of anti-inflammatory cytokines (e.g., by signaling of dendritic cells and induction of regulatory T-cells), triggering an immune response, and performing any other suitable function that adjusts a subject's microbiome away from a state of dysbiosis. However, probiotic therapies and/or prebiotic therapies can be configured in any suitable manner.

In another specific example, therapies can include medical-device based therapies (e.g., associated with human behavior modification, associated with treatment of disease-related conditions, etc.).

In variations, the therapy model is preferably based upon data from a large population of subjects, which can include the population of subjects from which the microbiome diversity datasets are derived in Block S110, where microbiome composition and/or functional features or states of health, prior exposure to and post exposure to a variety of therapeutic measures, are well characterized. Such data can be used to train and validate the therapy provision model, in identifying therapeutic measures that provide desired outcomes for subjects based upon different nasal-related characterizations. In variations, support vector machines, as a supervised machine learning algorithm, can be used to generate the therapy provision model. However, any other suitable machine learning algorithm described above can facilitate generation of the therapy provision model.

Additionally or alternatively, the therapy model can be derived in relation to identification of a “normal” or baseline microbiome composition and/or functional features, as assessed from subjects of a population of subjects who are identified to be in good health. Upon identification of a subset of subjects of the population of subjects who are characterized to be in good health (e.g., using features of the characterization process), therapies that modulate microbiome compositions and/or functional features toward those of subjects in good health can be generated in Block S140. Block S140 can thus include identification of one or more baseline microbiome compositions and/or functional features (e.g., one baseline microbiome for each of a set of demographic characteristics), and potential therapy formulations and therapy regimens that can shift microbiomes of subjects who are in a state of dysbiosis toward one of the identified baseline microbiome compositions and/or functional features. The therapy model can, however, be generated and/or refined in any other suitable manner.

Microorganism compositions associated with probiotic therapies and/or prebiotic therapies (e.g., associated with probiotic therapies determined by a therapy model applied by a therapy facilitation system, etc.) can include microorganisms that are culturable (e.g., able to be expanded to provide a scalable therapy) and/or non-lethal (e.g., non-lethal in their desired therapeutic dosages). Furthermore, microorganism compositions can include a single type of microorganism that has an acute or moderated effect upon a subject's microbiome. Additionally or alternatively, microorganism compositions can include balanced combinations of multiple types of microorganisms that are configured to cooperate with each other in driving a subject's microbiome toward a desired state. For instance, a combination of multiple types of bacteria in a probiotic therapy can include a first bacteria type that generates products that are used by a second bacteria type that has a strong effect in positively affecting a subject's microbiome. Additionally or alternatively, a combination of multiple types of bacteria in a probiotic therapy can include several bacteria types that produce proteins with the same functions that positively affect a subject's microbiome.

Probiotic and/or prebiotic compositions can be naturally or synthetically derived. For instance, in one application, a probiotic composition can be naturally derived from fecal matter or other biological matter (e.g., of one or more subjects having a baseline microbiome composition and/or functional features, as identified using the characterization process and the therapy model). Additionally or alternatively, probiotic compositions can be synthetically derived (e.g., derived using a benchtop method) based upon a baseline microbiome composition and/or functional features, as identified using the characterization process and the therapy model. In variations, microorganism agents that can be used in probiotic therapies can include one or more of: yeast (e.g., Saccharomyces boulardii), gram-negative bacteria (e.g., E. coli Nissle), gram-positive bacteria (e.g., Bifidobacteria bifidum, Bifidobacteria infantis, Lactobacillus rhamnosus, Lactococcus lactis, Lactobacillus plantarum, Lactobacillus acidophilus, Lactobacillus casei, Bacillus polyfermenticus, etc.), and any other suitable type of microorganism agent. However, probiotic therapies, prebiotic therapies and/or other suitable therapies can include any suitable combination of microorganisms associated with any suitable taxa described herein, and/or therapies can be configured in any suitable manner.

Block S140 can include executing, storing, retrieving, and/or otherwise processing one or more therapy models for determining one or more therapies. Processing one or more therapy models is preferably based on microbiome features. For example, generating a therapy model can based on microbiome features associated with one or more nasal-related conditions, therapy-related aspects such as therapy efficacy in relation to microbiome characteristics, and/or other suitable data. Additionally or alternatively, processing therapy models can be based on any suitable data. In an example, processing a therapy model can include determining one or more therapies for a user based on one or more therapy models, user microbiome features (e.g., inputting user microbiome feature values into the one or more therapy models, etc.), supplementary data (e.g., prior knowledge associated with therapies such as in relation to microorganism-related metabolization; user medical history; user demographic data, such as describing demographic characteristics; etc.), and/or any other suitable data. However, processing therapy models can be based on any suitable data in any suitable manner.

Nasal-related characterization models can include one or more therapy models. In an example, determining one or more nasal-related characterizations (e.g., for one or more users, for one or more nasal-related conditions, etc.), can include determining one or more therapies, such as based on one or more therapy models (e.g., applying one or more therapy models, etc.) and/or other suitable data (e.g., microbiome features such as user microbiome features, microorganism dataset such as user microorganism datasets, etc.). In a specific example, determining one or more nasal-related characterizations can include determining a first nasal-related characterization for a user (e.g., describing propensity for one or more nasal-related conditions; etc.); and determining a second nasal-related characterization for the user based on the first nasal-related characterization (e.g., determining one or more therapies, such as for recommendation to a user, based on the propensity for one or more nasal-related conditions; etc.). In a specific example, a nasal-related characterization can include both propensity-related data (e.g., diagnostic data; associated microbiome composition, function, diversity, and/or other characteristics; etc.) and therapy-related data (e.g., recommended therapies; potential therapies; etc.). However, nasal-related characterizations can include any suitable data (e.g., any combination of data described herein, etc.).

Processing therapy models can include processing a plurality of therapy models. For example, different therapy models can be processed for different therapies (e.g., different models for different individual therapies; different models for different combinations and/or categories of therapies, such as a first therapy model for determining consumable therapies and a second therapy model for determining psychological-associated therapies; etc.). In an example, different therapy models can be processed for different nasal-related conditions, (e.g., different models for different individual nasal-related conditions; different models for different combinations and/or categories of nasal-related conditions, etc.). Additionally or alternatively, processing a plurality of therapy models can be performed for (e.g., based on; processing different therapy models for; etc.) any suitable types of data and/or entities. However, processing a plurality of therapy models can be performed in any suitable manner, and determining and/or applying one or more therapy models can be performed in any suitable manner.

### 3.4 PROCESSING A USER BIOLOGICAL SAMPLE

Embodiments of the method 100 can additionally or alternatively include Block S150, which can include processing one or more biological samples from a user (e.g., biological samples from different collection sites of the user, etc.). Block S150 can function to facilitate generation of a microorganism dataset for a user, such as for use in deriving inputs for the characterization process (e.g., for generating a nasal-related characterization for the user, such as through applying one or more nasal-related characterization models, etc.). As such, Block S150 can include receiving, processing, and/or analyzing one or more biological samples from one or more users (e.g., multiple biological samples for the same user over time, different biological samples for different users, etc.). In Block S150, the biological sample is preferably generated from the user and/or an environment of the user in a non-invasive manner. In variations, non-invasive manners of sample reception can use any one or more of: a permeable substrate (e.g., a swab configured to wipe a region of a user's body, toilet paper, a sponge, etc.), a non-permeable substrate (e.g., a slide, tape, etc.) a container (e.g., vial, tube, bag, etc.) configured to receive a sample from a region of a user's body, and any other suitable sample-reception element. In a specific example, the biological sample can be collected from one or more of the user's nose, skin, genitals, mouth, and gut (e.g., through stool samples, etc.) in a non-invasive manner (e.g., using a swab and a vial). However, the biological sample can additionally or alternatively be received in a semi-invasive manner or an invasive manner. In variations, invasive manners of sample reception can use any one or more of: a needle, a syringe, a biopsy element, a lance, and any other suitable instrument for collection of a sample in a semi-invasive or invasive manner. In specific examples, samples can include blood samples, plasma/serum samples (e.g., to enable extraction of cell-free DNA), and tissue samples.

In the above variations and examples, the biological sample can be taken from the body of the user without facilitation by another entity (e.g., a caretaker associated with a user, a health care professional, an automated or semi-automated sample collection apparatus, etc.), or can alternatively be taken from the body of the user with the assistance of another entity. In one example, where the biological sample is taken from the user without facilitation by another entity in the sample extraction process, a sample-provision kit can be provided to the user. In the example, the kit can include one or more swabs for sample acquisition, one or more containers configured to receive the swab(s) for storage, instructions for sample provision and setup of a user account, elements configured to associate the sample(s) with the user (e.g., barcode identifiers, tags, etc.), and a receptacle that allows the sample(s) from the user to be delivered to a sample processing operation (e.g., by a mail delivery system). In another example, where the biological sample is extracted from the user with the help of another entity, one or more samples can be collected in a clinical or research setting from the user (e.g., during a clinical appointment). The biological sample can, however, be received from the user in any other suitable manner.

Furthermore, processing and analyzing biological samples (e.g., to generate a user microorganism dataset; etc.) from the user is preferably performed in a manner similar to that of one of the embodiments, variations, and/or examples described in relation to Block S110 above, and/or any other suitable portions of embodiments of the method 100 and/or system 200. As such, reception and processing of the biological sample in Block S150 can be performed for the user using similar processes as those for receiving and processing biological samples used to perform the characterization processes of the method 100, such as in order to provide consistency of process. However, biological sample reception and/or processing in Block S150 can additionally or alternatively be performed in any other suitable manner.

### 3.5 DETERMINING A NASAL-RELATED CHARACTERIZATION

Embodiments of the method 100 can additionally or alternatively include Block S160, which can include determining, with one or more characterization processes (e.g., one or more characterization processes described in relation to Block S130, etc.), a nasal-related characterization for the user, such as based upon processing one or more microorganism dataset (e.g., user microorganism sequence dataset, microbiome composition dataset, microbiome functional diversity dataset; processing of the microorganism dataset to extract user microbiome features (e.g., extract feature values; etc.) that can be used to determine the one or more nasal-related characterizations; etc.) derived from one or more samples (e.g., nose site samples, etc.) of the user. Block S160 can function to characterize one or more aspects associated with a user (e.g., associated with the sample of the user; associated with user characteristics; associated with one or more nasal-related conditions; etc.), such as through extracting features from microbiome-derived data of the user, and using the features as inputs into the characterization process described in Block S130 above (e.g., using the user microbiome feature values as inputs into a nasal-related characterization model, etc.). In an example, Block S160 can include generating a nasal-related characterization for the user based on user microbiome features and a nasal-related condition model (e.g., generated in Block S130). Nasal-related characterizations can include any suitable type of characterizations described herein (e.g., environmental factor-related characterizations; characterizations for any suitable supplementary data types; nasal-related condition characterizations, such as for a combination of nasal-related conditions, a single nasal-related condition, and/or other suitable nasal-related conditions; etc.), users, collection sites, and/or other suitable entities. In examples, nasal-related characterizations can include one or more of: diagnoses (e.g., presence or absence of a nasal-related condition; etc.); risk (e.g., risk scores for developing and/or the presence of a nasal-related condition; information regarding nasal-related characterizations (e.g., symptoms, signs, triggers, associated conditions, etc.); comparisons (e.g., comparisons with other subgroups, populations, users, historic health statuses of the user such as historic microbiome compositions and/or functional diversities; comparisons associated with nasal-related conditions; etc.); therapy determinations; other suitable outputs associated with characterization processes; and/or any other suitable data.

In a variation, a nasal-related characterization can include a microbiome diversity score (e.g., in relation to microbiome composition, function, etc.) associated with (e.g., correlated with; negatively correlated with; positively correlated with; etc.) with one or more taxa (e.g., correlated with a nose microbiome, such as in relation to a set of environmental factors; etc.), a nose site, nasal-related conditions, and/or other suitable aspects. In examples, the nasal-related characterization can include microbiome diversity scores over time (e.g., calculated for a plurality of biological samples of the user collected over time), comparisons to microbiome diversity scores for other users, and/or any other suitable type of microbiome diversity score. However, processing microbiome diversity scores (e.g., determining microbiome diversity scores; using microbiome diversity scores to determine and/or provide therapies; etc.) can be performed in any suitable manner.

Determining a nasal-related characterization in Block S160 can include determining features and/or combinations of features associated with the microbiome composition and/or functional features of the user (e.g., determining feature values associated with the user, the feature values corresponding to microbiome features determined in Block S130, etc.), inputting the features into the characterization process, and receiving an output that characterizes the user as belonging to one or more of: a behavioral group, a gender group, a dietary group, a disease-state group, and;any other suitable group capable of being identified by the characterization process. Block S160 can additionally or alternatively include generation of and/or output of a confidence metric associated with the characterization of the user. For example, a confidence metric can be derived from the number of features used to generate the characterization, relative weights or rankings of features used to generate the characterization, measures of bias in the characterization process, and/or any other suitable parameter associated with aspects of the characterization process. However, leveraging user microbiome features can be performed in any suitable manner to determine any suitable nasal-related characterizations.

In some variations, features extracted from the microorganism dataset of the user can be supplemented with supplementary features (e.g., user supplementary feature values extracted from supplementary data collected for the user; such as survey-derived features, medical history-derived features, sensor data, etc.), where such data, the user microbiome data, and/or other suitable data can be used to further refine the characterization process of Block S130, Block S160, and/or other suitable portions of embodiments of the method 100.

Determining a nasal-related characterization preferably includes extracting and applying user microbiome features (e.g., user microbiome composition diversity features; user microbiome functional diversity features; extracting feature values; etc.) for the user (e.g., based on a user microorganism dataset), characterization models, and/or other suitable components, such as by employing processes described in Block S130, and/or by employing any suitable approaches described herein.

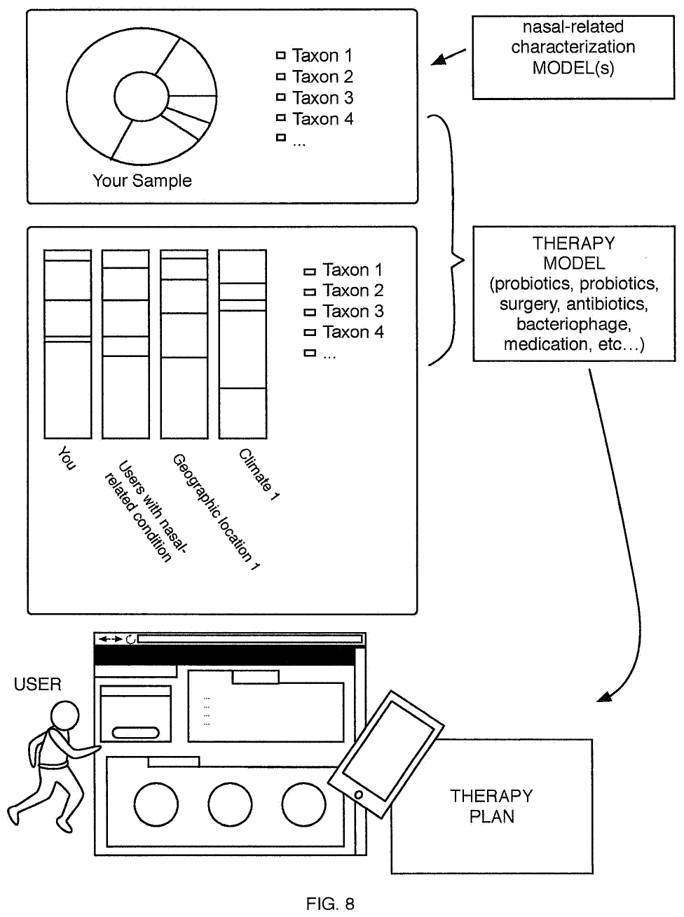

In variations, as shown in FIG. 8, Block S160 can include presenting nasal-related characterizations (e.g., information extracted from the characterizations; as part of facilitating therapeutic intervention; etc.), such as at a web interface, a mobile application, and/or any other suitable interface, but presentation of information can be performed in any suitable manner. However, the microorganism dataset of the user can additionally or alternatively be used in any other suitable manner to enhance the models of the method 100, and Block S160 can be performed in any suitable manner.

### 3.6 FACILITATING THERAPEUTIC INTERVENTION

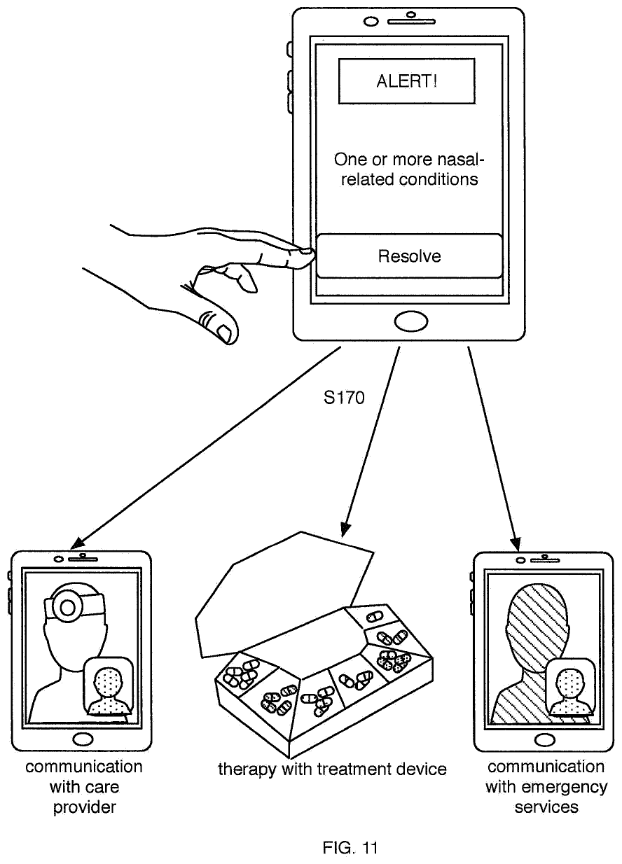

As shown in FIG. 11, embodiments of the method 100 can additionally or alternatively include Block S170, which can include facilitating therapeutic intervention (e.g., promoting therapies, providing therapies, facilitating provision of therapies, etc.) for one or more nasal-related conditions for one or more users (e.g., based upon a nasal-related characterization and/ or a therapy model). Block S170 can function to recommend, promote, provide, and/or otherwise facilitate therapeutic intervention in relation to one or more therapies for a user, such as to shift the microbiome composition and/or functional diversity of a user toward a desired equilibrium state (and/or otherwise improving a state of the nasal-related condition, etc.), such as in relation to one or more nasal-related conditions and/or reference nose microbiomes (e.g., healthy ranges of taxa for a nose microbiome; etc.). Block S170 can include provision of a customized therapy to the user according to their microbiome composition and functional features, where the customized therapy can include a formulation of microorganisms configured to correct dysbiosis characteristic of users having the identified characterization. As such, outputs of therapy determination can be used to directly promote a customized therapy formulation and regimen (e.g., dosage, usage instructions) to the user based upon a trained therapy model. Additionally or alternatively, therapy provision can include recommendation of available therapeutic measures configured to shift microbiome composition and/or functional features toward a desired state. In variations, therapies can include any one or more of: consumables, topical therapies (e.g., lotions, ointments, antiseptics, etc.), medication (e.g., medications associated with any suitable medication type and/or dosage, etc.), bacteriophages, environmental treatments, behavioral modification (e.g., diet modification therapies, stress-reduction therapies, physical activity-related therapies, etc.), diagnostic procedures, other medical-related procedures, and/or any other suitable therapies associated with nasal-related conditions. Consumables can include any one or more of: food and/or beverage items (e.g., probiotic and/or prebiotic food and/or beverage items, etc.), nutritional supplements (e.g., vitamins, minerals, fiber, fatty acids, amino acids, prebiotics, probiotics, etc.), consumable medications, and/or any other suitable therapeutic measure. In an example, providing one or more therapies and/or otherwise facilitating therapeutic intervention can include providing a recommendation for the one or more therapies to one or more users at one or more computing devices (e.g., at a user interface such as a web application, presented at the computing devices; etc.) associated with the one or more users.

For example, a combination of commercially available probiotic supplements can include a suitable probiotic therapy for the user according to an output of the therapy model. In another example, the method 100 can include determining a nasal-related condition risk for the user for the nasal-related condition based on a nasal-related condition model (e.g., and/or user microbiome features); and promoting a therapy to the user based on the nasal-related condition risk.

In a variation, facilitating therapeutic intervention can include promoting a diagnostic procedure (e.g., for facilitating detection of nasal-related conditions, which can motivate subsequent promotion of other therapies, such as for modulation of a user microbiome for improving a user health state associated with one or more nasal-related conditions; etc.). Diagnostic procedures can include any one or more of: medical history analyses, imaging examinations, cell culture tests, antibody tests, skin prick testing, patch testing, blood testing, challenge testing, performing portions of embodiments of the method 100, and/or any other suitable procedures for facilitating the detecting (e.g., observing, predicting, etc.) of nasal-related conditions. Additionally or alternatively, diagnostic device-related information and/or other suitable diagnostic information can be processed as part of a supplementary dataset (e.g., in relation to Block S120, where such data can be used in determining and/or applying characterization models, therapy models, and/or other suitable models; etc.), and/or collected, used, and/or otherwise processed in relation to any suitable portions of embodiments of the method 100 (e.g., administering diagnostic procedures for users for monitoring therapy efficacy in relation to Block 8180; etc.).

In a variation, Block S170 can include promoting a bacteriophage-based therapy. In more detail, one or more populations (e.g., in terms of colony forming units) of bacteriophages specific to a certain bacteria (or other microorganism) represented in the user can be used to down-regulate or otherwise eliminate populations of the certain bacteria. As such, bacteriophage-based therapies can be used to reduce the size(s) of the undesired population(s) of bacteria represented in the user. Complementarily, bacteriophage-based therapies can be used to increase the relative abundances of bacterial populations not targeted by the bacteriophage(s) used.

In a variation, facilitating therapeutic intervention (e.g., providing therapies, etc.) can include provision of notifications to a user regarding the recommended therapy, other forms of therapy, nasal-related characterizations, and/or other suitable data. In a specific example, providing a therapy to a user can include providing therapy recommendations (e.g., substantially concurrently with providing information derived from a nasal-related characterization for a user; etc.) and/or other suitable therapy-related information (e.g., therapy efficacy; comparisons to other individual users, subgroups of users, and/or populations of users; therapy comparisons; historic therapies and/or associated therapy-related information; psychological therapy guides such as for cognitive behavioral therapy; etc.), such as through presenting notifications at a web interface (e.g., through a user account associated with and identifying a user; etc.). Notifications can be provided to a user by way of an electronic device (e.g., personal computer, mobile device, tablet, wearable, head-mounted wearable computing device, wrist-mounted wearable computing device, etc.) that executes an application, web interface, and/or messaging client configured for notification provision. In one example, a web interface of a personal computer or laptop associated with a user can provide access, by the user, to a user account of the user, where the user account includes information regarding the user's nasal-related characterization, detailed characterization of aspects of the user's microbiome (e.g., in relation to correlations with nasal-related conditions; etc.), and/or notifications regarding suggested therapeutic measures (e.g., generated in Blocks S140 and/or S170, etc.). In another example, an application executing at a personal electronic device (e.g., smart phone, smart watch, head-mounted smart device) can be configured to provide notifications (e.g., at a display, haptically, in an auditory manner, etc.) regarding therapy suggestions generated by the therapy model of Block S170. Notifications and/or probiotic therapies can additionally or alternatively be provided directly through an entity associated with a user (e.g., a caretaker, a spouse, a significant other, a healthcare professional, etc.). In some further variations, notifications can additionally or alternatively be provided to an entity (e.g., healthcare professional) associated with a user, such as where the entity is able to facilitate provision of the therapy (e.g., by way of prescription, by way of conducting a therapeutic session, through a digital telemedicine session using optical and/or audio sensors of a computing device, etc.). Providing notifications and/or otherwise facilitating therapeutic, however, be performed in any suitable manner.

### 3.7 MONITORING THERAPY EFFECTIVENESS

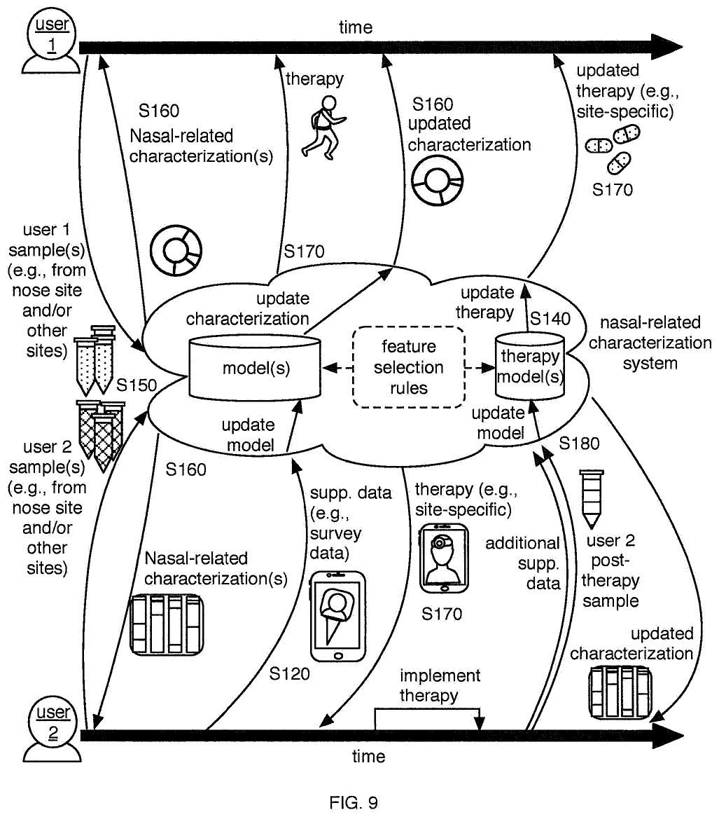

As shown in FIG. 9, the method 100 can additionally or alternatively include Block S180, which can include: monitoring effectiveness of one or more therapies and/or monitoring other suitable components (e.g., microbiome characteristics, etc.) for the user (e.g., based upon processing a series of biological samples from the user), over time. Block S180 can function to gather additional data regarding positive effects, negative effects, and/or lack of effectiveness of one or more therapies (e.g., suggested by the therapy model for users of a given characterization, etc.) and/or monitoring microbiome characteristics (e.g., to assess microbiome composition and/or functional features for the user at a set of time points, etc.).

Monitoring of a user during the course of a therapy promoted by the therapy model (e.g., by receiving and analyzing biological samples from the user throughout therapy, by receiving survey-derived data from the user throughout therapy) can thus be used to generate a therapy-effectiveness model for each characterization provided by the characterization process of Block S130, and each recommended therapy measure provided in Blocks S140 and S170.

In Block S180, the user can be prompted to provide additional biological samples, supplementary data, and/or other suitable data at one or more key time points of a therapy regimen that incorporates the therapy, and the additional biological sample(s) can be processed and analyzed (e.g., in a manner similar to that described in relation to Block S110) to generate metrics characterizing modulation of the user's microbiome composition and/or functional features. For instance, metrics related to one or more of: a change in relative abundance of one or more taxonomic groups represented in the user's microbiome at an earlier time point, a change in representation of a specific taxonomic group of the user's microbiome, a ratio between abundance of a first taxonomic group of bacteria and abundance of a second taxonomic group of bacteria of the user's microbiome, a change in relative abundance of one or more functional families in a user's microbiome, and any other suitable metrics can be used to assess therapy effectiveness from changes in microbiome composition and/or functional features. Additionally or alternatively, survey-derived data from the user, pertaining to experiences of the user while on the therapy (e.g., experienced side effects, personal assessment of improvement, behavioral modifications, symptom improvement, etc.) can be used to determine effectiveness of the therapy in Block S180. For example, the method 100 can include receiving a post-therapy biological sample from the user; collecting a supplementary dataset from the user, where the supplementary dataset describes user adherence to a therapy (e.g., a determined and promoted therapy) and/or other suitable user characteristics (e.g., behaviors, conditions, etc.); generating a post-therapy nasal-related characterization of the first user in relation to the nasal-related condition based on the nasal-related characterization model and the post-therapy biological sample; and promoting an updated therapy to the user for the nasal-related condition based on the post-therapy nasal-related characterization (e.g., based on a comparison between the post-therapy nasal-related characterization and a pre-therapy nasal-related characterization; etc.) and/or the user adherence to the therapy (e.g., modifying the therapy based on positive or negative results for the user microbiome in relation to the nasal-related condition; etc.). Additionally or alternatively, other suitable data (e.g., supplementary data describing user behavior associated with one or more nasal-related conditions; supplementary data describing a nasal-related condition such as observed symptoms; etc.) can be used in determining a post-therapy characterization (e.g., degree of change from pre- to post- therapy in relation to the nasal-related condition; etc.), updated therapies (e.g., determining an updated therapy based on effectiveness and/or adherence to the promoted therapy, etc.).

In an example, the method boo can include collecting supplementary data (e.g., survey-derived data; informing statuses of nasal-related conditions, such as in relation to symptom severity; etc.); determining the nasal-related characterization for the user based on the user microbiome features and the supplementary data; facilitating therapeutic intervention in relation to a therapy for the nasal-related condition (e.g., promoting the therapy to the user; etc.), based on the nasal-related characterization; collecting a post-therapy sample from the user (e.g., nose site sample; after facilitating the therapeutic intervention; etc.); collecting subsequent supplementary data (e.g., including at least one of second survey-derived data and device data; etc.); and determining a post-therapy nasal-related characterization for the user for the nasal-related condition based on the subsequent supplementary data and post-therapy user microbiome features associated with the post-therapy biological sample. In the example, the method 100 can include facilitating therapeutic intervention in relation to an updated therapy (e.g., a modification of the therapy; a different therapy; etc.) for the user for improving the nasal-related condition, based on the post-therapy nasal-related characterization, such as where the updated therapy can include at least one of a consumable, a device-related therapy, a surgical operation, a psychological-associated therapy, a behavior modification therapy, and an environmental factor modification therapy. In the example determining the post-therapy nasal-related characterization can include determining a comparison between microbiome characteristics of the user and reference microbiome characteristics corresponding to a user subgroup sharing at least one of a behavior and an environmental factor (and/or other suitable characteristic) associated with the nasal-related condition, based on the post-therapy microbiome features, and where facilitating therapeutic intervention in relation to the updated therapy can include presenting the comparison to the user for facilitating at least one of the behavior modification therapy and the environmental factor modification therapy and/or other suitable therapies. However, Block S180 can be performed in relation to additional biological samples, additional supplementary data, and/or other suitable additional data in any suitable manner.

Therapy effectiveness, processing of additional biological samples (e.g., to determine additional nasal-related characterizations, therapies, etc.), and/or other suitable aspects associated with continued biological sample collection, processing, and analysis in relation to nasal-related conditions can be performed at any suitable time and frequency for generating, updating, and/or otherwise processing models (e.g., characterization models, therapy models, etc.), and/or for any other suitable purpose (e.g., as inputs associated with other portions of embodiments of the method 100). However, Block S180 can be performed in any suitable manner.

### 4. SYSTEM

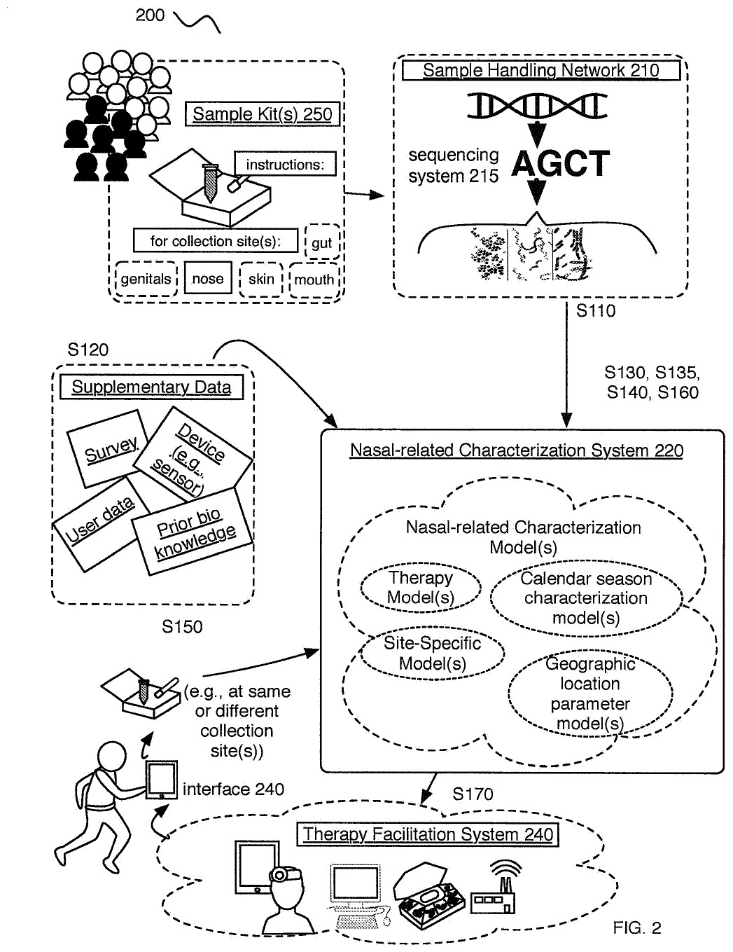

As shown in FIG. 2, embodiments of the system 200 (e.g., for characterizing a nasal-related condition) can include any one or more of: a handling system (e.g., a sample handling system, etc.) 210 operable to collect and/or process biological samples (e.g., collected by users and included in containers including pre-processing reagents; etc.) from one or more users (e.g., a human subject, patient, animal subject, environmental ecosystem, care provider, etc.) for facilitating determination of a microorganism dataset (e.g., microorganism genetic sequences; microorganism sequence dataset; etc.); a nasal-related characterization system 220 operable to determine microbiome features (e.g., microbiome composition features; microbiome functional features; diversity features; relative abundance ranges; such as based on a microorganism dataset and/or other suitable data; etc.), determine nasal-related characterizations (e.g., nasal-related condition characterizations, therapy-related characterizations, characterizations for users, etc.); and/or a therapy facilitation system 230 operable to facilitate therapeutic intervention (e.g., promote a therapy, etc.) for one or more nasal-related conditions (e.g., based on one or more nasal-related conditions; for improving one or more nasal-related conditions; etc.).

Embodiments of the system 200 can include one or more handling systems 210, which can function to receive and/or process (e.g., fragment, amplify, sequence, generate associated datasets, etc.) biological samples to transform microorganism nucleic acids and/or other components of the biological samples into data (e.g., genetic sequences that can be subsequently aligned and analyzed; microorganism datasets; etc.) for facilitating generation of nasal-related characterizations and/or therapeutic intervention. The handling system 210 can additionally or alternatively function to provide sample kits 250 (e.g., including sample, containers, instructions for collecting samples from one or more collection sites, etc.) to a plurality of users (e.g., in response to a purchase order for a sample kit 250), such as through a mail delivery system. The handling system 210 can include one or more sequencing systems 215 (e.g., next-generation sequencing systems, sequencing systems for targeted amplicon sequencing, sequencing-by-synthesis techniques, capillary sequencing technique, Sanger sequencing, pyrosequencing techniques, nanopore sequencing techniques, etc.) for sequencing one or more biological samples (e.g., sequencing microorganism nucleic acids from the biological samples, etc.), such as in generating microorganism data (e.g., microorganism sequence data, other data for microorganism datasets, etc.). Next-generation sequencing systems (e.g., next-generation sequencing platforms, etc.) can include any suitable sequencing systems (e.g., sequencing platforms, etc.) for one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), amplicon-associated sequencing (e.g., targeted amplicon sequencing), sequencing-by-synthesis, tunnelling currents sequencing, sequencing by hybridization, mass spectrometry sequencing, microscopy-based techniques, and/or any suitable next-generation sequencing technologies. Additionally or alternatively, sequencing systems 215 can implement any one or more of capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

The handling system 210 can additionally or alternatively include a library preparation system operable to automatically prepare biological samples (e.g., fragment and amplify using primers compatible with genetic targets associated with the nasal-related condition) in a multiplex manner to be sequenced by a sequencing system; and/or any suitable components. The handling system 210 can perform any suitable sample processing techniques described herein. However, the handling system 210 and associated components can be configured in any suitable manner.

Embodiments of the system 200 can include one or more nasal-related characterization systems 220, which can function to determine, analyze, characterize, and/or otherwise process microorganism datasets (e.g., based on processed biological samples leading to microorganism genetic sequences; alignments to reference sequences; etc.), microbiome features (e.g., individual variables; groups of variables; features relevant for phenotypic prediction, for statistical description; variables associated with a sample obtained from an individual; variables associated with nasal-related conditions; variables describing fully or partially, in relative or absolute quantities the sample's microbiome composition and/or functionality; etc.), models, and/or other suitable data for facilitating nasal-related characterization and/or therapeutic intervention. In examples, the nasal-related characterization system 220 can identify data associated with the information of the features that statistically describe the differences between samples associated with one or more nasal-related conditions (e.g., samples associated with presence, absence, risk of, propensity for, and/or other aspects related to nasal-related conditions etc.), such as where the differing analyses can provide complementing views into the features differentiating the different samples (e.g., differentiating the subgroups associated with presence or absence of a condition, etc.). In a specific example, individual predictors, a specific biological process, and/or statistically inferred latent variables can provide complementary information at different levels of data complexity to facilitate varied downstream opportunities in relation to characterization, diagnosis, and/or treatment. In another specific example, the nasal-related characterization system 220 process supplementary data for performing one or more characterization processes.

The nasal-related characterization system 220 can include, generate, apply, and/or otherwise process nasal-related characterization models, which can include any one or more of nasal-related condition models for characterizing one or more nasal-related conditions (e.g., determining propensity of one or more nasal-related conditions for one or more users, etc.), therapy models for determining therapies, and/or any other suitable models for any suitable purposes associated with the embodiments of the system 200 and/or method 100. In a specific example, the nasal-related characterization system 220 can generate and/or apply a therapy model (e.g., based on cross-condition analyses, etc.) for identifying and/or characterizing a therapy used to treat one or more nasal-related conditions. Different nasal-related characterization models (e.g., different combinations of nasal-related characterization models; different models applying different analytical techniques; different inputs and/or output types; applied in different manners such as in relation to time and/or frequency; etc.) can be applied (e.g., executed, selected, retrieves, stored, etc.) based on one or more of: supplementary data (e.g., different models for predicting different types of supplementary data such as different environmental factors, such as different models for different calendar seasons in predicting calendar season parameters, etc.), nasal-related conditions (e.g., using different nasal-related characterization models depending on the nasal-related condition or conditions being characterized, such as where different nasal-related characterization models possess differing levels of suitability for processing data in relation to different nasal related conditions and/or combinations of conditions, etc.), users (e.g., different nasal-related characterization models based on different user data and/or characteristics, demographic characteristics, genetics, environmental factors, etc.), nasal-related characterizations (e.g., different nasal-related characterization models for different types of characterizations, such as a therapy-related characterization versus a diagnosis-related characterization, such as for identifying relevant microbiome composition versus determining a propensity score for a nasal-related condition; etc.), therapies (e.g., different nasal-related characterization models for monitoring efficacy of different therapies, etc.), body sites (e.g., different nasal-related characterization models for processing microorganism datasets corresponding to biological samples from different sample collection sites; etc.), and/or any other suitable components. However, nasal-related characterization models can be tailored and/or used in any suitable manner for facilitating nasal related characterization and/or therapeutic intervention.

The nasal-related characterization system 220 can preferably determine site-specific nasal-related characterizations (e.g., site-specific analyses). In examples, the nasal-related characterization system 220 can generating and/or apply different site-specific nasal-related characterization models. In specific examples, different site-specific nasal-related characterization models can be generated and/or can be applied based on different microbiome features, such as site-specific features associated with the one or more body sites that the site-specific nasal-related characterization model is associated with (e.g., using nose site-specific features derived from samples collected at nose collection sites of subjects, and/or correlated with one or more nasal-related conditions, such as for generating a nose site-specific nasal-related characterization model that can be applied for determining characterizations based on user samples collected at user nose sites; etc.). Site-specific nasal-related characterization models, site-specific features, samples, site-specific therapies, and/or other suitable entities (e.g., able to be associated with a body site, etc.) are preferably associated with at least one body site (e.g., corresponding to a sample collection site; etc.) including one or more of a nose site, gut site (e.g., characterizable based on stool samples, etc.), skin site, genital site, mouth site, and/or any suitable body region. In examples, different nasal-related characterization models can be tailored to different types of inputs, outputs, nasal-related characterizations, nasal-related conditions (e.g., different phenotypic measures that need to be characterized), and/or any other suitable entities. However, site-specific nasal-related characterizations can be configured in any manner and determined in any manner by a nasal-related characterization system 220 and/or other suitable components.

Nasal-related characterization models, other models, other components of embodiments of the system 200, and/or suitable portions of embodiments of the method 100 (e.g., characterization processes, determining microbiome features, determining nasal-related characterizations, etc.) can employ analytical techniques including any one or more of: univariate statistical tests, multivariate statistical tests, dimensionality reduction techniques, artificial intelligence approaches (e.g., machine learning approaches, etc.), performing pattern recognition on data (e.g., identifying correlations between nasal-related conditions and microbiome features; etc.), fusing data from multiple sources (e.g., generating characterization models based on microbiome data and/or supplementary data from a plurality of users associated with one or more nasal-related conditions, such as based on microbiome features extracted from the data; etc.), combination of values (e.g., averaging values, etc.), compression, conversion (e.g., digital-to-analog conversion, analog-to-digital conversion), performing statistical estimation on data (e.g. ordinary least squares regression, non-negative least squares regression, principal components analysis, ridge regression, etc.), wave modulation, normalization, updating (e.g., of characterization models and/or therapy models based on processed biological samples over time; etc.), ranking (e.g., microbiome features; therapies; etc.), weighting (e.g., microbiome features; etc.), validating, filtering (e.g., for baseline correction, data cropping, etc.), noise reduction, smoothing, filling (e.g., gap filling), aligning, model fitting, binning, windowing, clipping, transformations, mathematical operations (e.g., derivatives, moving averages, summing, subtracting, multiplying, dividing, etc.), data association, multiplexing, demultiplexing, interpolating, extrapolating, clustering, image processing techniques, other signal processing operations, other image processing operations, visualizing, and/or any other suitable processing operations.

Artificial intelligence approaches can include any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.) reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), an ensemble method (e.g., boosting, boostrapped aggregation, AdaBoost, stacked generalization, gradient boosting machine method, random forest method, etc.), and/or any suitable artificial intelligence approach. However, data processing can be employed in any suitable manner.

The nasal-related characterization system 220 can perform cross-condition analyses for a plurality of nasal-related conditions (e.g., generating multi-condition characterizations based on outputs of different nasal-related characterization models, such as multi-condition microbiome features; etc.). For example, the nasal-related characterization system can characterize relationships between nasal-related conditions based on microorganism data, microbiome features, and/or other suitable microbiome characteristics of users associated with (e.g., diagnosed with, characterized by, etc.) a plurality of nasal-related conditions. In a specific example, cross-condition analyses can be performed based on characterizations for individual nasal-related conditions (e.g., outputs from nasal-related characterization models for individual nasal-related conditions, etc.). Cross-condition analyses can include identification of condition-specific features (e.g., associated exclusively with a single nasal-related condition, etc.), multi-condition features (e.g., associated with two or more nasal-related conditions, etc.), and/or any other suitable types of features. Cross-condition analyses can include determination of parameters informing correlation, concordance, and/or other similar parameters describing relationships between two or more nasal-related conditions, such as by evaluating different pairs of nasal-related conditions. However, the nasal-related characterization system and/or other suitable components can be configured in any suitable manner to facilitate cross-condition analyses (e.g., applying analytical techniques for cross-condition analysis purposes; generating cross-condition characterizations, etc.).

The nasal-related characterization system 220 can include a remote computing system (e.g., for applying nasal-related characterization models, etc.), but can additionally or alternatively include any suitable computing systems (e.g., local computing systems, user devices, handling system components, etc.). However, the nasal-related characterization system 220 can be configured in any suitable manner.

Embodiments of the system 200 can include one or more therapy facilitation systems 230, which can function to facilitate therapeutic intervention (e.g., promote one or more therapies, etc.) for one or more nasal-related conditions (e.g., facilitating modulation of a user microbiome composition and functional diversity for improving a state of the user in relation to one or more nasal-related conditions, etc.). The therapy facilitation system 230 can facilitate therapeutic intervention for any number of nasal-related conditions associated with any number of body sites (e.g., corresponding to any suitable number of collection sites of samples; etc.), such as based on site-specific characterizations (e.g., multi-site characterizations associated with a plurality of body sites; etc.), multi-condition characterizations, other characterizations, and/or any other suitable data. The therapy facilitation system 230 can include any one or more of: a communications system (e.g., to communicate therapy recommendations, selections, discouragements, and/or other suitable therapy-related information to a computing device (e.g., user device and/or care provider device; mobile device; smart phone; desktop computer; at a website, web application, and/or mobile application accessed by the computing device; etc.); to enable telemedicine between a care provider and a subject in relation to a nasal-related condition; etc.), an application executable on a user device (e.g., indicating microbiome composition and/or functionality for a user; etc.), a medical device (e.g., a biological sampling device, such as for collecting samples from different collection sites; medication provision devices; surgical systems; etc.), a user device (e.g., biometric sensors), and/or any other suitable component. One or more therapy facilitation systems 230 can be controllable, communicable with, and/or otherwise associated with the nasal-related characterization system 220. For example, the nasal-related characterization system 220 can generate characterizations of one or more nasal-related conditions for the therapy facilitation system 230 to present (e.g., transmit, communicate, etc.) to a corresponding user (e.g., at an interface 240, etc.). In another example, the therapy facilitation system 230 can update and/or otherwise modify an application and/or other software of a device (e.g., user smartphone) to promote a therapy (e.g., promoting, at a to-do list application, lifestyle changes for improving a user state associated with one or more nasal-related conditions, etc.). However, the therapy facilitation system 230 can be configured in any other manner.

As shown in FIG. 11, embodiments of the system 200 can additionally or alternatively include an interface 240, which can function to improve presentation of microbiome characteristics, nasal-related condition information (e.g., propensity metrics; therapy recommendations; comparisons to other users; other characterizations; etc.), and/or specific information (e.g., any suitable data described herein) associated with (e.g., included in, related to, derivable from, etc.) one or more nasal-related characterizations. In examples, the interface 240 can present nasal-related condition information including a microbiome composition (e.g., taxonomic groups; relative abundances; etc.), functional diversity (e.g., relative abundance of genes associated with particular functions, and propensity metrics for one or more nasal-related conditions, such as relative to user groups sharing a demographic characteristic (e.g., smokers, exercisers, users on different dietary regimens, consumers of probiotics, antibiotic users, groups undergoing particular therapies, etc.). However, the interface 240 can be configured in any suitable manner.

While the components of embodiments of the system 200 are generally described as distinct components, they can be physically and/or logically integrated in any manner. For example, a computing system (e.g., a remote computing system, a user device, etc.) can implement portions and/or all of the nasal-related characterization system 220 (e.g., apply a microbiome-related condition model to generate a characterization of nasal-related conditions for a user, etc.) and the therapy facilitation system 230 (e.g., facilitate therapeutic intervention through presenting insights associated with microbiome composition and/or function; presenting therapy recommendations and/or information; scheduling daily events at a calendar application of the smartphone to notify the user in relation to therapies for improving nasal-related, etc). In an example, embodiments of the system 200 can omit a therapy facilitation system 230. However, the functionality of embodiments of the system 200 can be distributed in any suitable manner amongst any suitable system components. However, the components of embodiments of the system 200 can be configured in any suitable manner

### 5. OTHER

Embodiments of the method 100 can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

Embodiments of the method 100 and/or system 200 can include every combination and permutation of the various system components and the various method processes, including any variants (e.g., embodiments, variations, examples, specific examples, figures, etc.), where portions of embodiments of the method 100 and/or processes described herein can be performed asynchronously (e.g., sequentially), concurrently (e.g., in parallel), or in any other suitable order by and/or using one or more instances, elements, components of, and/or other aspects of the system 200 and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method 100 and/or system 200 can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method 100, system 200, and/or variants without departing from the scope defined in the claims.

We claim:

1. A method for nasal-related characterization associated with microorganisms, the method comprising:
determining a microorganism sequence dataset associated with a set of subjects, based on microorganism nucleic acids from samples collected from nose sites of the set of subjects;
determining a set of microbiome composition features based on the microorganism sequence dataset;
generating a nasal-related characterization model based on the set of microbiome composition features and supplementary data associated with the set of subjects; and
determining a nasal-related characterization associated with a user based on the nasal-related characterization model and a user sample collected at a nose site of the user.

2. The method of claim 1, wherein the set of microbiome composition features is associated with at least one of Abiotrophia, Achromobacter, Acinetobacter, Actinobacillus, Actinomyces, Aggregatibacter, Alistipes, Alloprevotella, Anaerococcus, Anaerostipes, Anoxybacillus, Aquabacterium, Arthrobacter, Atopobium, Bacillus, Bacteroides, Bergeyella, Bifidobacterium, Blautia, Bradyrhizobium, Brevibacterium, Brevundimonas, Burkholderia, Campylobacter, Capnocytophaga, Caulobacter, Centipeda, Chryseobacterium, Collinsella, Corynebacterium, Deinococcus, Delftia, Dermabacter, Dialister, Dolosigranulum, Dorea, Enterobacter, Faecalibacterium, Finegoldia, Flavobacterium, Fusicatenibacter, Fusobacterium, Gemella, Granulicatella, Haemophilus, Herbaspirillum, Hydrogenophilus, Klebsiella, Kluyvera, Kocuria, Lactobacillus, Lactococcus, Lautropia, Leptotrichia, Malassezia, Megasphaera, Meiothermus, Methylobacterium, Micrococcus, Moraxella, Mycobacterium, Negativicoccus, Neisseria, Novosphingobium, Ochrobactrum, Pantoea, Parabacteroides, Parvimonas, Pelomonas, Peptoniphilus, Peptostreptococcus, Phyllobacterium, Porphyromonas, Prevotella, Propionibacterium, Pseudobutyrivibrio, Pseudomonas, Ralstonia, Rhizobium, Roseburia, Rothia, Sarcina, Shinella, Sphingomonas, Staphylococcus, Stenotrophomonas, Streptococcus, Veillonella, Parasutterella, Rhodopseudomonas, Xanthomonas, Mesorhizobium, Facklamia, Kingella, Rhodobacter, Lysinibacillus, Dermacoccus, and Cardiobacterium.

3. The method of claim 2, wherein the set of microbiome composition features comprises at least one relative abundance feature for the at least one of Abiotrophia, Achromobacter, Acinetobacter, Actinobacillus, Actinomyces, Aggregatibacter, Alistipes, Alloprevotella, Anaerococcus, Anaerostipes, Anoxybacillus, Aquabacterium, Arthrobacter, Atopobium, Bacillus, Bacteroides, Bergeyella, Bifidobacterium, Blautia, Bradyrhizobium, Brevibacterium, Brevundimonas, Burkholderia, Campylobacter, Capnocytophaga, Caulobacter, Centipeda, Chryseobacterium, Collinsella, Corynebacterium, Deinococcus, Delftia, Dermabacter, Dialister, Dolosigranulum, Dorea, Enterobacter, Faecalibacterium, Finegoldia, Flavobacterium, Fusicatenibacter, Fusobacterium, Gemella, Granulicatella, Haemophilus, Herbaspirillum, Hydrogenophilus, Klebsiella, Kluyvera, Kocuria, Lactobacillus, Lactococcus, Lautropia, Leptotrichia, Malassezia, Megasphaera, Meiothermus, Methylobacterium, Micrococcus, Moraxella, Mycobacterium, Negativicoccus, Neisseria, Novosphingobium, Ochrobactrum, Pantoea, Parabacteroides, Parvimonas, Pelomonas, Peptoniphilus, Peptostreptococcus, Phyllobacterium, Porphyromonas, Prevotella, Propionibacterium, Pseudobutyrivibrio, Pseudomonas, Ralstonia, Rhizobium, Roseburia, Rothia, Sarcina, Shinella, Sphingomonas, Staphylococcus, Stenotrophomonas, Streptococcus, Veillonella, Parasutterella, Rhodopseudomonas, Xanthomonas, Mesorhizobium, Facklamia, Kingella, Rhodobacter, Lysinibacillus, Dermacoccus, and Cardiobacterium.

4. The method of claim 2, wherein determining the nasal-related characterization comprises determining a calendar season parameter associated with the user sample collected at the nose site of the user, based on the nasal-related characterization model and the user sample.

5. The method of claim 4, wherein the nasal-related characterization model comprises a calendar season characterization machine learning model, wherein generating the nasal-related characterization model comprises training the calendar season characterization machine learning model based on the set of microbiome composition features and calendar seasons associated with the samples collected from the nose sites of the set of subjects, and wherein determining the calendar season parameter comprises determining the calendar season parameter based on the calendar season characterization machine learning model and the user sample collected at the nose site of the user.

6. The method of claim 4, wherein determining the calendar season parameter comprises determining at least one of a spring season prediction and a winter season prediction, associated with the sample, and wherein the user microbiome composition features are associated with at least one of: Parasutterella, Rhodopseudomonas, Xanthomonas, Mesorhizobium, Facklamia, Kingella, Rhodobacter, Lysinibacillus, Dermacoccus, and Cardiobacterium.

7. The method of claim 4, wherein the supplementary data comprises ages of the set of subjects, wherein generating the nasal-related characterization model comprises generating the nasal-related characterization model based on the set of microbiome composition features and the ages of the set of subjects, and wherein determining the calendar season parameter comprises determining the calendar season parameter based on the nasal-related characterization model, the user sample collected at the nose site of the user, and an age of the user.

8. The method of claim 7, wherein the supplementary data associated with the set of subjects comprises at least one of geographic location, climate type, and sampling time, wherein generating the nasal-related characterization model comprises generating the nasal-related characterization model based on the set of microbiome composition features, the ages of the set of subjects, and the at least one of geographic location, climate type, and sampling time, and wherein determining the calendar season parameter comprises determining the calendar season parameter based on the nasal-related characterization model, the user sample collected at the nose site of the user, the age of the user, and at least one of user geographic location, climate type associated with the user geographic location, and user sampling time associated with the user sample collected at the nose site of the user.

9. The method of claim 2, wherein determining the nasal-related characterization comprises determining a geographic location parameter associated with the user sample, based on the nasal-related characterization model and the user sample.

10. The method of claim 1, wherein the nasal-related characterization model is associated with a nasal-related condition, and wherein determining the nasal related characterization comprises determining the nasal-related characterization for the user for the nasal-related condition, based on the nasal-related characterization model and the user sample collected at the nose site of the user.

11. The method of claim 10, further comprising providing a therapy to the user for facilitating improvement of the nasal-related condition, based on the nasal-related characterization.

12. The method of claim 1, wherein determining the microorganism sequence dataset associated with the set of subjects comprises determining the microorganism sequence dataset based on sequencing the microorganism nucleic acids with a next-generation sequencing system.

13. A method for nasal-related characterization associated with microorganisms, the method comprising:
collecting a sample from a user, wherein the sample is from a nose site of the user and comprises microorganism nucleic acids;
determining a microorganism dataset associated with the user based on the microorganism nucleic acids of the sample;
determining user microbiome composition features based on the microorganism dataset; and
determining a nasal-related characterization associated with the user based on the user microbiome composition features.

14. The method of claim 13, wherein the user microbiome composition features are associated with at least one of Abiotrophia, Achromobacter, Acinetobacter, Actinobacillus, Actinomyces, Aggregatibacter, Alistipes, Alloprevotella, Anaerococcus, Anaerostipes, Anoxybacillus, Aquabacterium, Arthrobacter, Atopobium, Bacillus, Bacteroides, Bergeyella, Bifidobacterium, Blautia, Bradyrhizobium, Brevibacterium, Brevundimonas, Burkholderia, Campylobacter, Capnocytophaga, Caulobacter, Centipeda, Chryseobacterium, Collinsella, Corynebacterium, Deinococcus, Delftia, Dermabacter, Dialister, Dolosigranulum, Dorea, Enterobacter, Faecalibacterium, Finegoldia, Flavobacterium, Fusicatenibacter, Fusobacterium, Gemella, Granulicatella, Haemophilus, Herbaspirillum, Hydrogenophilus, Klebsiella, Kluyvera, Kocuria, Lactobacillus, Lactococcus, Lautropia, Leptotrichia, Malassezia, Megasphaera, Meiothermus, Methylobacterium, Micrococcus, Moraxella, Mycobacterium, Negativicoccus, Neisseria, Novosphingobium, Ochrobactrum, Pantoea, Parabacteroides, Parvimonas, Pelomonas, Peptoniphilus, Peptostreptococcus, Phyllobacterium, Porphyromonas, Prevotella, Propionibacterium, Pseudobutyrivibrio, Pseudomonas, Ralstonia, Rhizobium, Roseburia, Rothia, Sarcina, Shinella, Sphingomonas, Staphylococcus, Stenotrophomonas, Streptococcus, Veillonella, Parasutterella, Rhodopseudomonas, Xanthomonas, Mesorhizobium, Facklamia, Kingella, Rhodobacter, Lysinibacillus, Dermacoccus, and Cardiobacterium.

15. The method of claim 14, wherein determining the nasal-related characterization associated with the user comprises determining the nasal-related characterization based on the user microbiome composition features and an age of the user.

16. The method of claim 13, wherein determining the nasal-related characterization comprises determining a calendar season parameter associated with the sample from the nose site of the user, based on the user microbiome composition features.

17. The method of claim 16, wherein determining the calendar season parameter comprises determining at least one of a spring season prediction and a winter season prediction, associated with the sample, and wherein the user microbiome composition features are associated with at least one of: Parasutterella, Rhodopseudomonas, Xanthomonas, Mesorhizobium, Facklamia, Kingella, Rhodobacter, Lysinibacillus, Dermacoccus, and Cardiobacterium.

18. The method of claim 13, wherein determining the nasal-related characterization for the user comprises determining the nasal-related characterization based on the user microbiome composition features and supplementary data associated with the user, wherein the supplementary data comprises at least one of geographic location, climate type, and sampling time.

19. The method of claim 13, wherein determining the nasal-related characterization associated with the user comprises determining a geographic location parameter associated with the sample from the nose site of the user, based on the user microbiome composition features.

20. The method of claim 13, wherein determining the nasal-related characterization comprises determining the nasal-related characterization for the user for a nasal related condition associated with the microorganisms, based on the user microbiome composition features.

21. The method of claim 20, further comprising facilitating therapeutic intervention in relation to a therapy for the user for facilitating improvement of the nasal-related condition, based on the nasal-related characterization.

22. The method of claim 13, wherein determining the nasal-related characterization comprises determining the nasal-related characterization associated with the user based on the user microbiome composition features and a nasal-related characterization machine learning model trained with a set of microbiome composition features and supplementary data associated with a set of subjects.
